(19) United States
(12) Patent Application Publication
(10) Pub. No.: US 2010/0267596 A1
(43) Pub. Date: Oct. 21, 2010

(54) METHOD FOR IMPROVING THE HYDROLYSIS STABILITY OF IONIC LIQUIDS

(75) Inventors: Georg Degen, Lorsch (DE); Veit Stegmann, Mannheim (DE); Klaus Ebel, Lampertheim (DE); Klemens Massonne, Bad Duerkheim (DE); Laszlo Szarvas, Ludwigshafen (DE); Uwe Vagt, Mannheim (DE); Matthias Maase, Mendham, NJ (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASE SE, Ludwigshafen (DE)

(21) Appl. No.: 12/747,372

(22) PCT Filed: Dec. 12, 2008
(86) PCT No.: PCT/EP08/67428

§ 371 Date: Jun. 10, 2010

(30) Foreign Application Priority Data
Dec. 14, 2007 (EP) 07150039.1

(51) Int. Cl. C07D 207/24; C02F 5/10; C09K 3/00; B01F 1/00; B01J 31/02; H01M 4/88; C09K 5/04; G01N 33/00; C07K 1/26

(52) U.S. Cl. 508/268; 252/180; 252/190; 252/194; 252/364; 502/168; 252/182.1; 252/67; 252/408.1; 204/450

(57) ABSTRACT

The present invention relates to a method for improving the hydrolysis stability of an ionic liquid (IL), in which at least one tertiary amine or one quaternary ammonium compound different from the ionic liquid (IL) is added to an ionic liquid (IL).

The present invention relates to a method for improving the hydrolysis stability of an ionic liquid (IL), in which at least one tertiary amine or one quaternary ammonium compound different from the ionic liquid (IL) is added to an ionic liquid (IL).

Ionic liquids display a series of interesting properties. They are thermally stable, nonflammable, have an extremely low, barely measurable vapor pressure, are mostly environmentally friendly, have a large liquidus range and have very good solvent properties for numerous substances. In addition, ionic liquids also have, owing to their purely ionic structure, interesting electrochemical properties such as the electrical conductivity which is frequently accompanied by a high electrochemical stability. Variation of the side chains of the cation and selection of suitable anions allows, for example, the solubility in water or organic solvents or the melting point to be determined freely to a substantial extent.

The molecular variety of ionic liquids makes it possible to use them in many industrial applications. Examples are extraction (e.g. isolation and purification of industrial gases, isolation and purification of hydrocarbons in the petrochemicals industry and in organic synthesis or the removal of toxic substances from wastewater), the sorption, drying, purification and storage of gases (e.g. in sorption air conditioning units), use as solvents (e.g. for organic synthesis), immobilization of catalysts, use as lubricant, hydraulic fluid or antistatic additive, use as electrolyte (e.g. in electroplating, in fuel cells, capacitors, sensor and battery technology, metal upgrading, photovoltaics or in electrochromic components), use as electroelastic material (e.g. in actuators), use for heat transport or heat storage (e.g. thermofluids or PCM media) or use as special analytical reagent (e.g. matrix materials, solvents for Karl-Fischer titration or media for protein crystallization or electrophoresis).

Owing to the need to match the properties of ionic liquids to specific applications, anions which are subject to hydrolysis under storage and/or use conditions are frequently employed in ionic liquids. This hydrolysis can, even when it occurs to only a small extent, substantially influence the chemical and physical properties of the ionic liquids. Examples are alteration of the melting point of the ionic liquid and the formation of corrosive hydrolysis products. Replacement of (partially) hydrolyzed ionic liquids is therefore often absolutely necessary.

WO 03022812 describes ionic liquids which have a compound of the formula [R—SO₄]⁻ as anion, where R is a linear or branched, saturated or unsaturated, aliphatic or alicyclic, functionalized or unfunctionalized alkyl radical having from 3 to 36 carbon atoms. These anions are, in contrast to methylsulfate or ethylsulfate anions, hydrolysis-stable in neutral aqueous solution. However, the ionic liquids in which these anions are present can only be obtained with increased difficulty.

It is therefore an object of the invention to provide a method by means of which the hydrolysis of customary, hydrolyzable anions of ionic liquids can be substantially slowed or prevented. This should result in longer operating lives of ionic liquids and/or, if the products of the hydrolysis of the anion have an increased corrosivity, a reduction in the damaging action on chemical compounds, chemical reactions and apparatuses which are brought into contact with the ionic liquids. Furthermore, the ionic liquids used should, if possible, be able to be disposed of thermally without formation of problematical combustion gases, be biodegradable and be able to be obtained easily.

It has now surprisingly been found that the addition of even small amounts of a tertiary amine and/or a quaternary ammonium compound different from the ionic liquid leads to a significant reduction in the decomposition of the anion of the ionic liquid, especially of sulfate anions, by hydrolysis.

The present invention therefore provides a method for improving the hydrolysis stability of an ionic liquid (IL), in which at least one tertiary amine and/or one quaternary ammonium compound different from the ionic liquid (IL) is added to the ionic liquid (IL).

For the purposes of the present patent application, ionic liquids are organic salts which are liquid at temperatures below 180° C. The ionic liquids preferably have a melting point of less than 180° C. The melting point is more preferably in the range from −50° C. to 150° C., more preferably in the range from −20° C. to 120° C. and even more preferably below 100° C.

Ionic liquids which are present in the liquid state at room temperature are described, for example, by K. N. Marsh et al., Fluid Phase Equilibria 219 (2004), 93-98 and J. G. Huddleston et al., Green Chemistry 2001, 3, 156-164.

Cations and anions are present in the ionic liquid. Within the ionic liquid, a proton or an alkyl radical can be transferred from the cation to the anion, resulting in two uncharged molecules. An equilibrium of anions, cations and uncharged molecules formed therefrom can thus be present in the ionic liquid used according to the invention.

For the purposes of the present invention, the expression “alkyl” comprises straight-chain or branched alkyl. It is preferably straight-chain or branched C₁-C₃₀-alkyl, in particular C₁-C₁₈-alkyl and very particularly preferably C₁-C₁₂-alkyl. Examples of alkyl groups are, in particular, methyl, ethyl, n-propyl, isopropyl, n-butyl, isobutyl, sec-butyl, tert-butyl, n-pentyl, isopentyl, 1-methylbutyl, tert-pentyl, neopentyl, n-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, n-heptyl, n-octyl, 1-methylheptyl, 2-ethylhexyl, 2,4,4-trimethyl-pentyl, 1,1,3,3-tetramethylbutyl, n-nonyl, n-decyl, n-undecyl, n-dodecyl, n-tridecyl, n-tetradecyl, n-pentadecyl, n-hexadecyl, n-heptadecyl, n-octadecyl and n-eicosyl.

The expression alkyl also comprises alkyl radicals whose carbon chain may be interrupted by one or more nonadjacent heteroatoms or heteroatom-comprising groups which are preferably selected from among —O—, —S—, —NR^{E}—, —PR^{E}—, —SiR^{E}R^{EE} and —SO₂—. R^{E} is preferably hydrogen, alkyl, cycloalkyl, heterocycloalkyl, aryl or hetaryl. R^{EE} is preferably hydrogen, alkyl, cycloalkyl, heterocycloalkyl or aryl.

Examples of alkyl radicals whose carbon chains may be interrupted by one or two nonadjacent heteroatoms —O— are the following:

methoxymethyl, diethoxymethyl, 2-methoxyethyl, 2-ethoxyethyl, 2-propoxyethyl, diethoxyethyl, 2-butoxyethyl, 2-octyloxyethyl, 2-methoxypropyl, 3-methoxypropyl, 3-ethoxypropyl, 3-propoxypropyl, 2-isopropoxyethyl, 2-butoxypropyl, 3-butoxypropyl, 4-methoxybutyl, 4-ethoxybutyl, 4-propoxybutyl, 6-methoxyhexyl, 3,6-dioxaheptyl (5-methoxy-3-oxapentyl), 3,6-dioxaoctyl (7-methoxy-4-oxaheptyl), 4,8-dioxanonyl (7-methoxy-4-oxaheptyl), 3,7-dioxaoctyl, 3,7-dioxanonyl, 4,7-dioxaoctyl, 4,7-dioxanonyl, 2- and 4-butoxybutyl, 4,8-dioxadecyl, 9-ethoxy-5-oxanonyl.

Examples of alkyl radicals whose carbon chains may be interrupted by three or more than three nonadjacent heteroatoms —O— also include oligooxyalkylenes and poly-oxyalkylenes, i.e. compounds having repeating units which are preferably selected from among (CH₂CH₂O)ₓ₁, (CH(CH₃)CH₂O)ₓ₂ and ((CH₂)₄O)ₓ₃, where x1, x2 and x3 are each, independently of one another, an integer from 0 to 100, preferably from 0 to 80, with the proviso that the sum of x1, x2 and x3 is at least 3. Preferably x1, x2 and x3 are each, independently of one another, an integer from 3 to 100, preferably from 3 to 80. The sum of x1, x2 and x3 is preferably an integer from 3 to 300, in particular from 3 to 100. In polyoxyalkylenes which have two or three different repeating units, the repeating units can be present in any order, i.e. the repeating units can be randomly distributed, alternating or arranged in blocks. Examples are 3,6,9-trioxadecyl, 3,6,9-trioxaundecyl, 3,6,9-trioxadodecyl, 4,8,12-trioxamidecyl (11-methoxy-4,8-dioxaundecyl), 4,8,12-trioxatetradecyl, 14-methoxy-5,10-dioxatetradecyl, 5,10,15-trioxaheptadecyl, 3,6,9,12-tetraoxamidecyl, 3,6,9,12-tetraoxatetradecyl, 4,8,12,16-tetraoxaheptadecyl (15-methoxy-4,8,12-trioxapentadecyl), 4,8,12,16-tetraoxaoctadecyl and the like.

Examples of alkyl radicals whose carbon chains may be interrupted by one or more, e.g. 1, 2, 3, 4 or more than 4, nonadjacent heteroatoms —S— are the following:

butylthiomethyl, 2-methylthioethyl, 2-ethylthioethyl, 2-propylthioethyl, 2-butylthioethyl, 2-dodecylthioethyl, 3-methylthiopropyl, 3-ethylthiopropyl, 3-propylthiopropyl, 3-butylthiopropyl, 4-methylthiobutyl, 4-ethylthiobutyl, 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-trithiadecyl, 3,6,9-trithiaundecyl, 3,6,9-trithiadodecyl, 3,6,9,12-tetrathiamidecyl and 3,6,9,12-tetrathiatetradecyl.

Examples of alkyl radicals whose carbon chains are interrupted by one or two nonadjacent heteroatom-comprising groups —NR^{E}— are the following:

2-monomethyl- and 2-monoethylaminoethyl, 2-dimethylaminoethyl, 3-methylamino-propyl, 2- and 3-dimethylaminopropyl, 3-monoisopropylaminopropyl, 2- and 4-monopropylaminobutyl, 2- and 4-dimethylaminobutyl, 6-methylaminohexyl, 6-dimethylaminohexyl, 6-methyl-3,6-diazaheptyl, 3,6-dimethyl-3,6-diazaheptyl, 3,6-diazaoctyl and 3,6-dimethyl-3,6-diazaoctyl.

Examples of alkyl radicals whose carbon chains may be interrupted by three or more nonadjacent heteroatom-comprising groups —NR^{E}— also include oligoalkylenimines and polyalkylenimines. What has been said above with regard to the polyoxyalkylenes applies analogously to polyalkylenimines, with the oxygen atom being replaced in each case by an NR^{E} group, where R^{a} is preferably hydrogen or C₁-C₄-alkyl. Examples are 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazamidecyl, 3,6,9,12-tetramethyl-3,6,9,12-tetraazamidecyl and the like.

Examples of alkyl radicals whose carbon chains are interrupted by one or more, e.g. 1 or 2, nonadjacent groups —SO₂— are 2-methylsulfonylethyl, 2-ethylsulfonylethyl, 2-propylsulfonylethyl, 2-isopropylsulfonylethyl, 2-butylsulfonylethyl, 2-methylsulfonyl-propyl, 3-methylsulfonylpropyl, 2-ethylsulfonylpropyl, 3-ethylsulfonylpropyl, 2-propyl-sulfonylpropyl, 3-propylsulfonylpropyl, 2-butylsulfonylpropyl, 3-butylsulfonylpropyl, 2-methylsulfonylbutyl, 4-methylsulfonylbutyl, 2-ethylsulfonylbutyl, 4-ethylsulfonylbutyl, 2-propylsulfonylbutyl, 4-propylsulfonylbutyl and 4-butylsulfonylbutyl.

The expression alkyl also comprises substituted alkyl radicals. Substituted alkyl groups can, depending on the length of the alkyl chain, have one or more (e.g. 1, 2, 3, 4, 5 or more than 5) substituents. These are preferably selected independently from among cycloalkyl, cycloalkyloxy, polycyclyl, polycyclyloxy, heterocycloalkyl, aryl, aryloxy, arylthio, hetaryl, halogen, hydroxy, SH, ═O, ═S, ═NR^{E}, COOH, carboxylate, SO₃H, sulfonate, NE¹E², nitro and cyano, where E¹ and E² are each, independently of one another, hydrogen, alkyl, cycloalkyl, heterocycloalkyl, aryl or hetaryl. Cycloalkyl, cycloalkyloxy, polycycloalkyl, polycycloalkyloxy, heterocycloalkyl, aryl and hetaryl substituents on the alkyl groups may in turn be unsubstituted or substituted; suitable substituents are those mentioned below for these groups.

What has been said above with regard to alkyl also implies in principle to the alkyl parts of alkoxy, alkylamino, dialkylamino, alkylthio (alkylsulfanyl), alkylsulfinyl, alkylsulfonyl, etc.

Suitable substituted alkyl radicals are the following:

Alkyl which is substituted by carboxy, e.g. carboxymethyl, 2-carboxyethyl, 3-carboxy-propyl, 4-carboxybutyl, 5-carboxypentyl, 6-carboxyhexyl, 7-carboxyheptyl, 8-carboxy-octyl, 9-carboxynonyl, 10-carboxydecyl, 12-carboxydodecyl and 14-carboxytetradecyl.
Alkyl which is substituted by SO₃H, e.g. sulfomethyl, 2-sulfoethyl, 3-sulfopropyl, 4-sulfobutyl, 5-sulfopentyl, 6-sulfohexyl, 7-sulfoheptyl, 8-sulfooctyl, 9-sulfononyl, 10-sulfodecyl, 12-sulfododecyl and 14-sulfotetradecyl.
Alkyl which is substituted by carboxylate, e.g. alkoxycarbonylalkyl, e.g. methoxycarbonylmethyl, ethoxycarbonylmethyl, n-butoxycarbonylmethyl, 2-methoxycarbonylethyl, 2-ethoxycarbonylethyl, 2-methoxycarbonylpropyl, 2-ethoxycarbonylpropyl, 2-(n-butoxycarbonyl)propyl, 2-(4-n-butoxycarbonyl)propyl, 3-methoxycarbonylpropyl, 3-ethoxycarbonylpropyl, 3-(n-butoxycarbonyl)propyl, 3-(4-n-butoxycarbonyl)propyl, aminocarbonylalkyl, e.g. aminocarbonylmethyl, aminocarbonylethyl, aminocarbonylpropyl and the like, alkylaminocarbonylalkyl such as methylaminocarbonylmethyl, methylaminocarbonylethyl, ethylcarbonylmethyl, ethylcarbonylethyl and the like or dialkylaminocarbonylalkyl such as dimethylaminocarbonylmethyl, dimethylaminocarbonylethyl, dimethylcarbonylpropyl, diethylaminocarbonylmethyl, diethylaminocarbonylethyl, diethylcarbonylpropyl and the like.
Alkyl which is substituted by hydroxy, e.g. 2-hydroxyethyl, 2-hydroxypropyl, 3-hydroxy-propyl, 3-hydroxybutyl, 4-hydroxybutyl, 2-hydroxy-2,2-dimethylethyl, 5-hydroxy-3-oxa-pentyl, 6-hydroxyhexyl, 7-hydroxy-4-oxaheptyl, 8-hydroxy-4-oxaoctyl, 8-hydroxy-3,6-dioxaoctyl, 9-hydroxy-5-oxanonyl, 11-hydroxy-4,8-dioxaundecyl, 11-hydroxy-3,6,9-trioxaundecyl, 14-hydroxy-5,10-dioxatetradecyl, 15-hydroxy-4,8,12-trioxapenta-decyl and the like.
Alkyl which is substituted by amino, e.g. 2-aminoethyl, 2-aminopropyl, 3-aminopropyl, 4-aminobutyl, 6-aminohexyl and the like.
Alkyl which is substituted by cyano, e.g. 2-cyanoethyl, 3-cyanopropyl, 3-cyanobutyl and 4-cyanobutyl;
Alkyl which is substituted by halogen as defined below, with some or all of the hydrogen atoms in the alkyl group being able to be replaced by halogen atoms, for example C₁-C₁₈-fluoroalkyl, e.g. trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl and the like, C₁-C₁₈-chloroalkyl, e.g. chloromethyl, dichloromethyl, trichloromethyl, 2-chloroethyl, 2- and 3-chloropropyl, 2-, 3- and 4-chlorobutyl, 1,1-dimethyl-2-chloroethyl and the like, C₁-C₁₈-bromoalkyl, e.g. bromoethyl, 2-bromoethyl, 2- and 3-bromopropyl and 2-, 3- and 4-bromobutyl and the like.
Alkyl which is substituted by nitro, e.g. 2-nitroethyl, 2- and 3-nitropropyl and 2-, 3- and 4-nitrobutyl and the like.
Alkyl which is substituted by amino, e.g. 2-aminoethyl, 2-aminopropyl, 3-aminopropyl, 4-aminobutyl, 6-aminohexyl and the like.
Alkyl which is substituted by cycloalkyl, e.g. cyclopentylmethyl, 2-cyclopentylethyl, 3-cyclopentylpropyl, cyclohexylmethyl, 2-cyclohexylethyl, 3-cyclohexylpropyl and the like.
Alkyl which is substituted by ═O (oxo group), e.g. 2-oxopropyl, 2-oxobutyl, 3-oxobutyl, 1-methyl-2-oxopropyl, 2-oxopentyl, 3-oxopentyl, 1-methyl-2-oxobutyl, 1-methyl-3-oxo-butyl, 2-oxohexyl, 3-oxohexyl, 4-oxohexyl, 2-oxoheptyl, 3-oxoheptyl, 4-oxoheptyl, 4-oxoheptyl and the like.
Alkyl which is substituted by ═S (thioxo group), e.g. 2-thioxopropyl, 2-thioxobutyl, 3-thioxobutyl, 1-methyl-2-thioxopropyl, 2-thioxopentyl, 3-thioxopentyl, 1-methyl-2-thioxobutyl, 1-methyl-3-thioxobutyl, 2-thioxohexyl, 3-thioxohexyl, 4-thioxohexyl, 2-thioxoheptyl, 3-thioxoheptyl, 4-thioxoheptyl, 4-thioxoheptyl and the like.
Alkyl which is substituted by ═NR^{E}, preferably groups in which R^{E} is hydrogen or C₁-C₄-alkyl, e.g. 2-iminopropyl, 2-iminobutyl, 3-iminobutyl, 1-methyl-2-iminopropyl, 2-imino-pentyl, 3-iminopentyl, 1-methyl-2-iminobutyl, 1-methyl-3-iminobutyl, 2-iminohexyl, 3-iminohexyl, 4-iminohexyl, 2-iminoheptyl, 3-iminoheptyl, 4-iminoheptyl, 4-iminoheptyl, 2-methyliminopropyl, 2-methyliminobutyl, 3-methyliminobutyl, 1-methyl-2-methyl-iminopropyl, 2-methyliminopentyl, 3-methyliminopentyl, 1-methyl-2-methyliminobutyl, 1-methyl-3-methyliminobutyl, 2-methyliminohexyl, 3-methyliminohexyl, 4-methylimino-hexyl, 2-methyliminoheptyl, 3-methyliminoheptyl, 4-methyliminoheptyl, 4-methylimino-heptyl, 2-ethyliminopropyl, 2-ethyliminobutyl, 3-ethyliminobutyl, 1-methyl-2-ethylimino-propyl, 2-ethyliminopentyl, 3-ethyliminopentyl, 1-methyl-2-ethyliminobutyl, 1-methyl-3-ethyliminobutyl, 2-ethyliminohexyl, 3-ethyliminohexyl, 4-ethyliminohexyl, 2-ethylimino-heptyl, 3-ethyliminoheptyl, 4-ethyliminoheptyl, 4-ethyliminoheptyl, 2-propyliminopropyl, 2-propyliminobutyl, 3-propyliminobutyl, 1-methyl-2-propyliminopropyl, 2-propylimino-pentyl, 3-propyliminopentyl, 1-methyl-2-propyliminobutyl, 1-methyl-3-propyliminobutyl, 2-propyliminohexyl, 3-propyliminohexyl, 4-propyliminohexyl, 2-propyliminoheptyl, 3-propyliminoheptyl, 4-propyliminoheptyl, 4-propyliminoheptyl and the like.
Alkyl which is substituted by aryl (“arylalkyl”) has at least one unsubstituted or substituted aryl group as defined below. Suitable substituents on the aryl group are those mentioned below. The alkyl group in “arylalkyl” may bear at least one further substituent as defined above and/or be interrupted by one or more nonadjacent heteroatoms or heteroatom-comprising groups which are selected from among —O—, —S—, —NR^{E}— and —SO₂—. Arylalkyl is preferably phenyl-C₁-C₁₀-alkyl, particularly preferably phenyl-C₁-C₄-alkyl, e.g. benzyl, 1-phenethyl, 2-phenethyl, 1-phenprop-1-yl, 2-phenprop-1-yl, 3-phenprop-1-yl, 1-phenbut-1-yl, 2-phenbut-1-yl, 3-phenbut-1-yl, 4-phenbut-1-yl, 1-phenbut-2-yl, 2-phenbut-2-yl, 3-phenbut-2-yl, 4-phenbut-2-yl, 1-(phenmeth)eth-1-yl, 1-(phenmethyl)-1-(methyl)eth-1-yl or -(phenmethyl)-1-(methyl)prop-1-yl; preferably benzyl and 2-phenethyl.
Alkoxy is an alkyl group bound via an oxygen atom. Examples of alkoxy are: methoxy, ethoxy, n-propoxy, 1-methylethoxy, butoxy, 1-methylpropoxy, 2-methylpropoxy, 1,1-dimethylethoxy, n-pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethyl-propoxy, 1,2,2-trimethylpropoxy, 1-ethyl-1-methylpropoxy or 1-ethyl-2-methylpropoxy, hexoxy and also R^{A}O—(CH₂CH₂CH₂CH₂O)ₙ—CH₂CH₂CH₂CH₂O— where R^{A} is hydrogen or C₁-C₄-alkyl, preferably hydrogen, methyl or ethyl, and n is from 0 to 10, preferably from 0 to 3.
Alkylthio (alkylsulfanyl) is an alkyl group bound via a sulfur atom. Examples of alkylthio are methylthio, ethylthio, propylthio, butylthio, pentylthio and hexylthio.
Alkylsulfinyl is an alkyl group bound via an S(═O) group.
Alkylsulfonyl is an alkyl group bound via an S(═O)₂ group.

For the purposes of the present invention, the expression “alkenyl” comprises straight-chain and branched alkenyl groups which, depending on the length of the chain, may have one or more double bonds (e.g. 1, 2, 3, 4 or more than 4). Preference is given to C₂-C₁₈-, particularly preferably C₂-C₁₂-alkenyl groups. The expression “alkenyl” also comprises substituted alkenyl groups which may bear one or more (e.g. 1, 2, 3, 4, 5 or more than 5) substituents. Suitable substituents are, for example, selected from among ═O, ═S, ═NR^{E}, cycloalkyl, cycloalkyloxy, polycyclyl, polycyclyloxy, heterocycloalkyl, aryl, aryloxy, arylthio, hetaryl, halogen, hydroxy, SH, COOH, carboxylate, SO₃H, sulfonate, alkylsulfinyl, alkylsulfonyl, NE³E⁴, nitro and cyano, where E³ and E⁴ are each, independently of one another, hydrogen, alkyl, cycloalkyl, heterocycloalkyl, aryl or hetaryl.

The expression “alkenyl” also comprises alkenyl radicals whose carbon chain may be interrupted by one or more nonadjacent heteroatoms or heteroatom-comprising groups which are preferably selected from among —O—, —S—, —NR^{E}— and —SO₂—.

Alkenyl is then, for example, ethenyl (vinyl), 1-propenyl, 2-propenyl, 1-methylethenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like.

For the purposes of the present invention, the expression “cycloalkyl” comprises both unsubstituted and substituted monocyclic saturated hydrocarbon groups which generally have from 3 to 12 ring carbons, preferably C₃-C₁₂-cycloalkyl groups, such as cyclopropyl, cyclobutyl, cyclopentyl, cyclohexyl, cycloheptyl, cyclooctyl, cyclononyl, cyclodecyl, cycloundecyl or cyclododecyl, in particular C₅-C₁₂-cycloalkyl. Suitable substituents are generally selected from among alkyl, the substituents mentioned above for alkyl groups, alkoxy and alkylthio. Substituted cycloalkyl groups may have one or more (e.g. 1, 2, 3, 4, 5 or more than 5) substituents, and in the case of halogen the cycloalkyl radical may be partially or fully substituted by halogen.

Examples of cycloalkyl groups are cyclopentyl, 2- and 3-methylcyclopentyl, 2- and 3-ethylcyclopentyl, chloropentyl, dichloropentyl, dimethylcyclopentyl, cyclohexyl, 2-, 3- and 4-methylcyclohexyl, 2-, 3- and 4-ethylcyclohexyl, 3- and 4-propylcyclohexyl, 3- and 4-isopropylcyclohexyl, 3- and 4-butylcyclohexyl, 3- and 4-sec-butylcyclohexyl, 3- and 4-tert-butylcyclohexyl, chlorohexyl, dimethylcyclohexyl, diethylcyclohexyl, methoxy-cyclohexyl, dimethoxycyclohexyl, diethoxycyclohexyl, butoxycyclohexyl, methylthiocyclohexyl, chlorocyclohexyl, dichlorocyclohexyl, cycloheptyl, 2-, 3- and 4-methylcycloheptyl, 2-, 3- and 4-ethylcycloheptyl, 3- and 4-propylcycloheptyl, 3- and 4-isopropylcycloheptyl, 3- and 4-butylcycloheptyl, 3- and 4-sec-butylcycloheptyl, 3- and 4-tert-butylcycloheptyl, cyclooctyl, 2-, 3-, 4- and 5-methylcyclooctyl, 2-, 3-, 4- and 5-ethylcyclooctyl, 3-, 4- and 5-propylcyclooctyl, partially fluorinated cycloalkyl and perfluorinated cycloalkyl of the formula CₙF_{2(n-a)-(1-b)}H_{2a-b} where n=5 to 12, 0<=a<=n and b=0 or 1.

Cycloalkyloxy is a cycloalkyl group as defined above which is bound via oxygen.

The expression “cycloalkenyl” comprises unsubstituted and substituted, monounsaturated or doubly unsaturated hydrocarbon groups having from 3 to 5, up to 8, up to 12, preferably from 5 to 12, ring carbons, e.g. cyclopent-1-en-1-yl, cyclopent-2-en-1-yl, cyclopent-3-en-1-yl, cyclohex-1-en-1-yl, cyclohex-2-en-1-yl, cyclohex-3-en-1-yl, cyclohexa-2,5-dien-1-yl and the like. Suitable substituents are those mentioned above for cycloalkyl.

Cycloalkenyloxy is a cycloalkenyl group as defined above which is bound via oxygen.

For the purposes of the present invention, the expression “polycyclyl” comprises in the broadest sense compounds which comprise at least two rings, regardless of how these rings are linked. They can be carbocyclic and/or heterocyclic rings. The rings can be saturated or unsaturated. The rings can be linked via a single or double bond (“multinuclear compounds”), joined by fusion (“fused ring systems”) or bridged (“bridged ring systems”, “cage compounds”). Preferred polycyclic compounds are bridged ring systems and fused ring systems. Fused ring systems can be aromatic, hydroaromatic and cyclic compounds linked by fusion (fused-on). Fused ring systems comprise two, three or more than three rings. Depending on the way in which the rings are linked, a distinction is made in the case of fused ring systems between ortho-fusion, i.e. each ring shares an edge or two atoms with each neighboring ring, and peri-fusion in which one carbon atom belongs to more than two rings. Among fused ring systems, preference is given to ortho-fused ring systems. For the purposes of the present invention, bridged ring systems include ones which are neither included among multinuclear ring systems nor among fused ring systems and in which at least two ring atoms belong to at least two different rings. In the case of bridged ring systems, a distinction is made, according to the number of ring-opening reactions which are formally required to obtain an open-chain compound, between bicyclo, tricyclo and tetracyclo compounds, etc., which comprise two, three, four, etc., rings. The expression “bicycloalkyl” comprises bicyclic hydrocarbon radicals having preferably from 5 to 10 carbon atoms, e.g. bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like. The expression “bicycloalkenyl” comprises monounsaturated, bicyclic hydrocarbon radicals having preferably from 5 to 10 carbon atoms, e.g. bicyclo[2.2.1]hept-2-en-1-yl.

For the purposes of the present invention, the expression “aryl” comprises monocyclic or polycyclic aromatic hydrocarbon radicals which may be unsubstituted or substituted. Aryl is generally hydrocarbon radicals having from 6 to 10, up to 14, up to 18, preferably from 6 to 10, ring carbons. Aryl is preferably unsubstituted or substituted phenyl, naphthyl, anthracenyl, phenanthrenyl, naphthacenyl, chrysenyl, pyrenyl, etc., and particularly preferably phenyl or naphthyl. Substituted aryls can, depending on the number and size of ring systems in them, have one or more (e.g. 1, 2, 3, 4, 5 or more than 5) substituents. These are preferably selected independently from among alkyl, alkoxy, cycloalkyl, cycloalkyloxy, heterocycloalkyl, aryl, aryloxy, arylthio, hetaryl, halogen, hydroxy, SH, alkylthio, alkylsulfinyl, alkylsulfonyl, COOH, carboxylate, SO₃H, sulfonate, NE⁵E⁶, nitro and cyano, where E⁵ and E⁶ are each, independently of one another, hydrogen, alkyl, cycloalkyl, cycloalkyloxy, polycyclylyl, polycyclyloxy, heterocycloalkyl, aryl, aryloxy or hetaryl. Aryl is particularly preferably phenyl, which if it is substituted can generally bear 1, 2, 3, 4 or 5, preferably 1, 2 or 3, substituents.

Aryl which bears one or more radicals is, for example, 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butyl-phenyl, 2-, 3- and 4-tert-butylphenyl, 2,4-, 2,5-, 3,5- and 2,6-di-tert-butylphenyl, 2,4,6-tri-tert-butylphenyl and 2-, 3-, 4-dodecylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxy-phenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl, 2-, 3- and 4-butoxyphenyl, 2-, 3-, 4-hexyloxy-phenyl; 2-, 3-, 4-chlorophenyl, 2,4-, 2,5-, 3,5- and 2,6-dichlorophenyl, trichlorophenyl, 2-, 3-, 4-fluorophenyl, 2,4-, 2,5-, 3,5- and 2,6-difluorophenyl, trifluorophenyl, for example 2, 4,6-trifluorophenyl, tetrafluorophenyl, pentafluorophenyl, 2-, 3- and 4-cyanophenyl; 2-nitrophenyl, 4-nitrophenyl, 2,4-dinitrophenyl, 2,6-dinitrophenyl; 4-dimethylaminophenyl; 4-acetylphenyl; methoxyethylphenyl, ethoxymethylphenyl; methylthiophenyl, isopropylthiophenyl or tert-butylthiophenyl; methylnaphthyl; isopropylnaphthyl or ethoxynaphthyl. Examples of substituted aryl in which two substituents bound to adjacent carbon atoms of the aryl ring form a fused ring or fused ring system are indenyl and fluorenyl.

For the purposes of the present invention, the expression “aryloxy” refers to aryl bound via an oxygen atom.

For the purposes of the present invention, the expression “arylthio” refers to aryl bound via a sulfur atom.

For the purposes of the present invention, the expression “heterocycloalkyl” refers to nonaromatic, unsaturated or fully saturated, cycloaliphatic groups which generally have from 5 to 8 ring atoms, preferably 5 or 6 ring atoms, and in which 1, 2 or 3 of the ring carbons have been replaced by heteroatoms selected from among oxygen, nitrogen, sulfur and a group —NR^{E}— and which are unsubstituted or substituted by one or more, for example 1, 2, 3, 4, 5 or 6, C₁-C₆-alkyl groups. Examples of such heterocycloaliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothienyl, dihydrothienyl, tetrahydrofuranyl, dihydrofuranyl, tetrahydropyranyl, 1,2-oxazolin-5-yl, 1,3-oxazolin-2-yl and dioxanyl. Nitrogen-comprising heterocycloalkyl can in principle be bound either via a carbon atom or via a nitrogen atom.

For the purposes of the present invention, the expression “heteroaryl (hetaryl)” comprises unsubstituted or substituted, heteroaromatic, monocyclic or polycyclic groups which generally have from 5 to 14 ring atoms, preferably 5 or 6 ring atoms, and in which 1, 2 or 3 of the ring carbons have been replaced by one, two, three or four heteroatoms selected from among O, N, —NR^{E}— and S, e.g. furyl, thienyl, oxazolyl, isoxazolyl, thiazolyl, isothiazolyl, benzofuranyl, benzthiazolyl, benzimidazolyl, pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl, where these heterocycloaromatic groups can, if they are substituted, generally bear 1, 2 or 3 substituents. The substituents are generally selected from among C₁-C₆-alkyl, C₁-C₆-alkoxy, hydroxy, carboxy, halogen and cyano.

5- to 7-membered nitrogen-comprising heterocycloalkyl or heteroaryl radicals which may optionally comprise further heteroatoms are, for example, pyrrolyl, pyrazolyl, imidazolyl, triazolyl, pyrrolidinyl, pyrazolinyl, pyrazolidinyl, imidazolinyl, imidazolidinyl, pyridinyl, pyridazinyl, pyrimidinyl, pyrazinyl, triazinyl, piperidinyl, piperazinyl, oxazolyl, isooxazolyl, thiazolyl, isothiazolyl, indolyl, quinolinyl, isoquinolinyl or quinaldinyl which may be unsubstituted or substituted as mentioned above.

Halogen is fluorine, chlorine, bromine or iodine.

For the purposes of the present invention, carboxylate and sulfonate are preferably derivatives of a carboxylic acid function or a sulfonic acid function, in particular a metal carboxylate or sulfonate function, a carboxylic ester or sulfonic ester function or a carboxamide or sulfonamide function. These include, for example, esters with C₁-C₄-alkanols such as methanol, ethanol, n-propanol, isopropanol, n-butanol, sec-butanol and tert-butanol.

For the purposes of the present invention, the expression “acyl” refers to alkanoyl, hetaroyl or aroyl groups generally having from 1 to 11, preferably from 2 to 8, carbon atoms, for example the formyl, acetyl, propanoyl, butanoyl, pentanoyl, hexanoyl, heptanoyl, 2-ethylhexanoyl, 2-propylheptanoyl, benzoyl or naphthoyl group.

The radicals E¹ and E², E³ and E⁴, E⁵ and E⁶ are selected independently from among hydrogen, alkyl, cycloalkyl and aryl. The groups NE¹E², NE³E⁴ and NE⁵E⁶ are preferably N,N-dimethylamino, N,N-diethylamino, N,N-dipropylamino, N,N-diisopropyl-amino, N,N-di-n-butylamino, N,N-di-tert-butylamino, N,N-dicyclohexylamino or N,N-diphenylamino.

For the purposes of the present invention, suitable ionic liquids are selected from among salts of the general formula (I)

[A]⁺(1/n)*[Y]ⁿ⁻ (I),

where [A]⁺ is a quaternary ammonium cation and (1/n)*[Y]ⁿ⁻ is an anion equivalent of an anion bearing n charges, where n is an integer from 1 to 3.

Compounds suitable for the formation of the cation [A]⁺ of ionic liquids are described, for example, in DE 102 02 838 A1. These compounds preferably comprise at least one nitrogen atom, particularly preferably from 1 to 10 nitrogen atoms, in particular from 1 to 5 nitrogen atoms, very particularly preferably from 1 to 3 nitrogen atoms and especially 1 or 2 nitrogen atoms. The latter nitrogen compounds can comprise further heteroatoms such as oxygen, sulfur or phosphorus atoms.

Nitrogen atoms are, for example, suitable carriers of the positive charge in the cation of ionic liquids. In the synthesis of the ionic liquids, a cation can firstly be produced by quaternization of the nitrogen atom of, for instance, an amine or nitrogen heterocycle. Quaternization can be effected by protonation of the nitrogen atom. Depending on the protonation reagent used, salts having different anions are obtained. In cases in which it is not possible to form the desired anion in the quaternization itself, this can be brought about in a further step of the synthesis. Starting from, for example, an ammonium halide, the halide can be reacted with a Lewis acid, forming a complex anion from the halide and Lewis acid. As an alternative, replacement of a halide ion by the desired anion is possible. This can be achieved by addition of a metal salt with precipitation of the metal halide formed, by means of an ion exchanger or by displacement of the halide ion by a strong acid (with liberation of the hydrogen halide). Suitable methods are described, for example, in Angew. Chem. 2000, 112, pp. 3926-3945, and the references cited therein.

Preferred cations of the ionic liquids are compounds which have a molar mass of less than 1000 g/mol, very particularly preferably less than 600 g/mol and in particular less than 400 g/mol.

Further preferred cations of the ionic liquids are compounds which comprise at least one five- or six-membered heterocycle, in particular a five-membered heterocycle, which has at least one nitrogen atom and optionally an oxygen or sulfur atom; particular preference is given to compounds which comprise at least one five- or six-membered heterocycle having one, two or three nitrogen atoms and a sulfur or oxygen atom, very particularly preferably those having two nitrogen atoms. Further preference is given to aromatic heterocycles.

Accordingly, in a preferred embodiment of the method of the invention, the hydrolysis stability of an ionic liquid (IL) which has a heterocyclic cation will be increased.

For the purposes of the present invention, the term “heterocyclic” cation comprises both “heteroaromatic” cations and “partially or fully saturated heterocyclic cations”.

The term “heteroaromatic” cation comprises cations whose structure can be obtained, for example, by quaternization of a ring nitrogen of a “hetaryl” compound as defined above. Examples of five- or six-membered heteroaromatic cations are pyrazolium, oxazolium, isoxazolium, thiazolium, isothiazolium, imidazolium, 1,2,4-oxadiazolium, 1,2,4-thiadiazolium, 1,3,4-oxadiazolium, 1,3,4-thiadiazolium, pyrrolium, 1,2,3-triazolium, 1,2,4-triazolium, pyridinium, pyridazinium, pyrimidinium, 2-pyrazinium, 1,3,5-triazinium and 1,2,4-triazinium.

The term “partially or fully saturated” heterocyclic cation comprises cations whose structure can be obtained, for example, by quaternization of a ring nitrogen of a “heterocycloalkyl” compound as defined above. Examples of five- or six-membered saturated or partially unsaturated heterocyclic cations are pyrrolidinium, pyrazolidinium, oxazolidinium, isoxazolidinium, thiazolidinium, isothiazolidinium, imidazolidinium, 1,2,4-oxadiazolidinium, 1,2,4-thiadiazolidinium, 1,2,4-triazolidinium, 1,3,4-oxa-diazolidinium, 1,3,4-thiadiazolidinium, 1,3,4-triazolidinium, 2-pyrrolinium, 3-pyrrolinium, 2-isoxazolinium, 3-isoxazolinium, 4-isoxazolinium, 2-isothiazolinium, 3-isothiazolinium, 4-isothiazolinium, 2,3-dihydropyrazolium, 3,4-dihydropyrazolium, 4,5-dihydro-pyrazolium, 2,3-dihydrooxazolium, 3,4-dihydrooxazolium, piperidinium, hexahydro-pyridazinium, hexahydropyrimidinium, piperazinium, 1,3,5-hexahydrotriazinium and 1,2,4-hexahydrotriazinium.

The ionic liquid IL used according to the invention preferably has at least one cation selected from among the following compounds of the formulae (IV.a) to (IV.v),

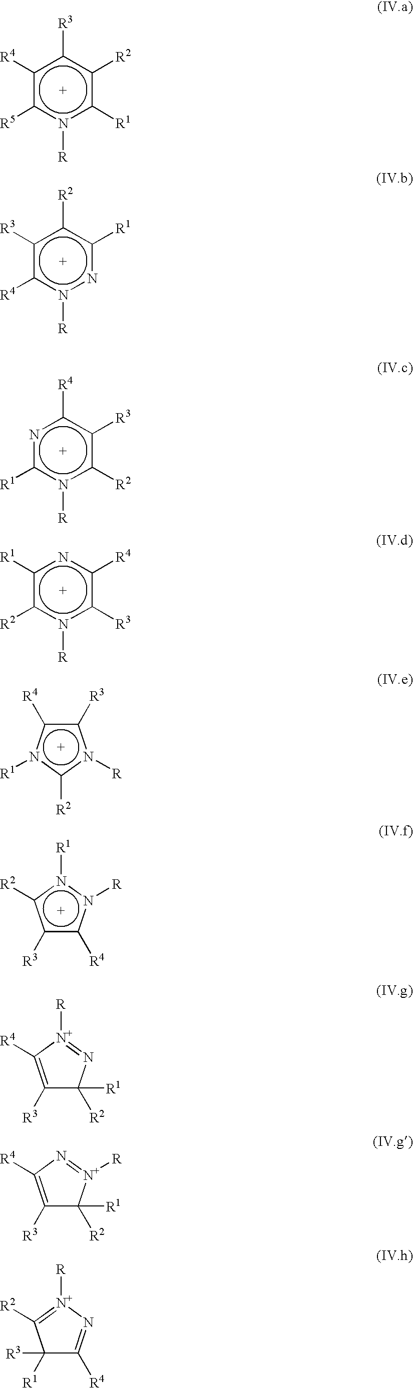

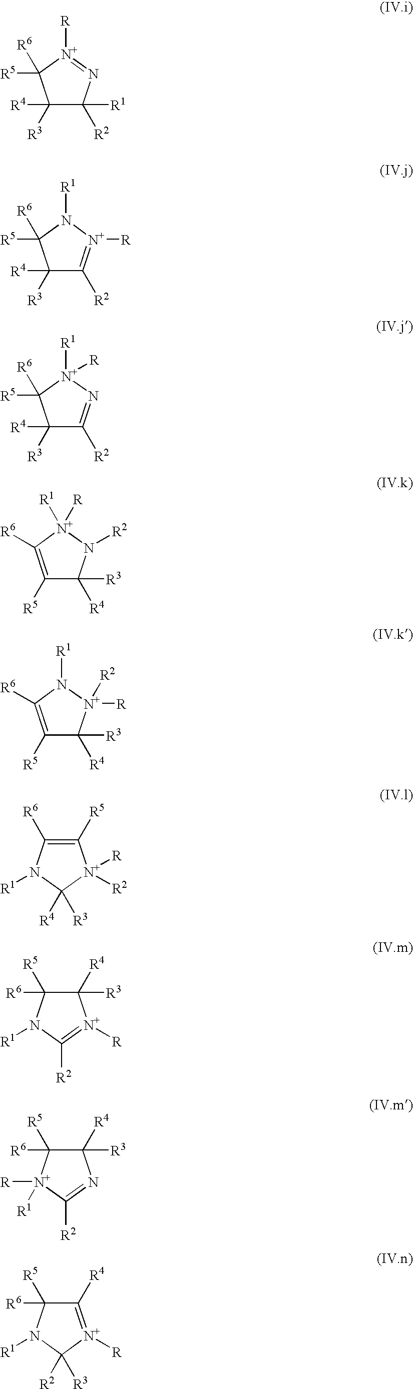

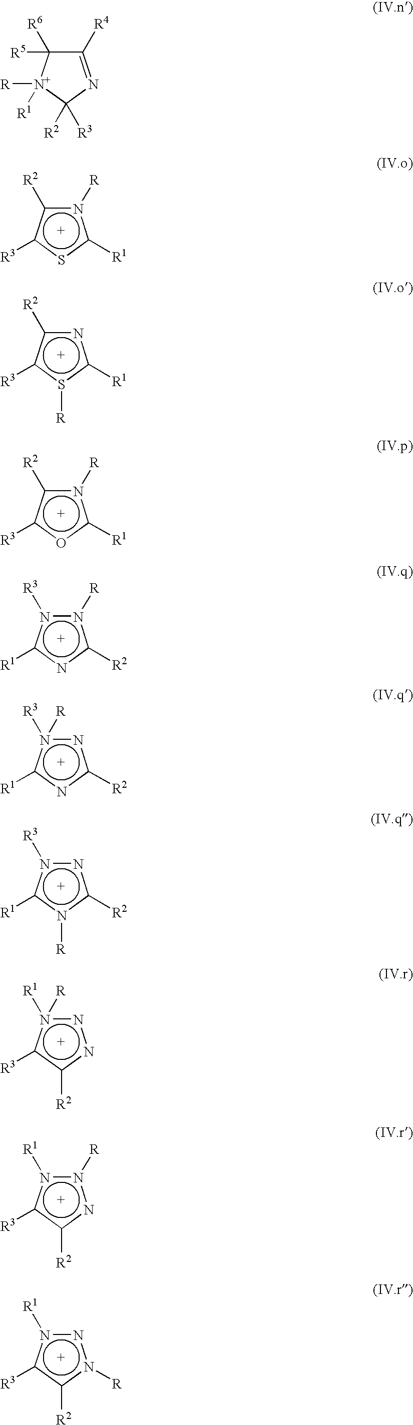

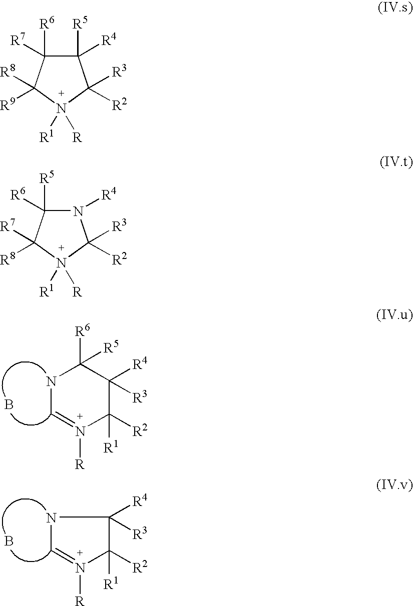

and oligomers comprising these structures, where
R is hydrogen, alkyl, alkenyl, cycloalkyl, cycloalkenyl, polycyclyl, heterocycloalkyl, aryl or heteroaryl;
radicals R¹, R², R³, R⁴, R⁵, R⁶, R⁷, R⁸ and R⁹ which are bound to a ring carbon are each, independently of one another, hydrogen, a sulfo group, COOH, carboxylate, sulfonate, acyl, alkoxycarbonyl, cyano, halogen, hydroxyl, SH, nitro, NE¹E², alkyl, alkoxy, alkylthio, alkylsulfinyl, alkylsulfonyl, alkenyl, cycloalkyl, cycloalkyloxy, cycloalkenyl, cycloalkenyloxy, polycyclyl, polycyclyloxy, heterocycloalkyl, aryl, aryloxy or heteroaryl, where E¹ and E² are each, independently of one another, hydrogen, alkyl, cycloalkyl, heterocycloalkyl, aryl or hetaryl,
radicals R¹, R², R³, R⁴, R⁵, R⁶, R⁷, R⁸ and R⁹ which are bound to a ring heteroatom are each hydrogen, SO₃H, NE¹E², alkyl, alkoxy, alkenyl, cycloalkyl, cycloalkenyl, polycyclyl, heterocycloalkyl, aryl or heteroaryl, where E¹ and E² are each, independently of one another, hydrogen, alkyl, cycloalkyl, heterocycloalkyl, aryl or hetaryl, or
two adjacent radicals R¹ to R⁹ together with the ring atoms to which they are bound may also form at least one fused, saturated, unsaturated or aromatic ring or a ring system having from 1 to 30 carbon atoms, where the ring or the ring system may comprise from 1 to 5 nonadjacent heteroatoms or heteroatom-comprising groups and the ring or the ring system may be unsubstituted or substituted,
two geminal radicals R¹ to R⁹ may together also be ═O, ═S or ═NR^{b}, where R^{b} is hydrogen, alkyl, cycloalkyl, aryl or heteroaryl,
and in the compounds of the formula (IV.u), R¹ and R³ or R³ and R⁵ may together also represent the bond part of a double bond between the ring atoms bearing these radicals,
B in the compounds of the formulae (IV.u) and (IV.v) together with the CN group to which it is bound forms a 4- to 8-membered, saturated or unsaturated or aromatic ring which may optionally be substituted and/or may optionally have further heteroatoms or heteroatom-comprising groups and/or may comprise further fused, saturated, unsaturated or aromatic carbocycles or heterocycles.

With regard to the general meaning of the abovementioned radicals carboxylate, sulfonate, acyl, alkoxycarbonyl, halogen, NE¹E², alkyl, alkoxy, alkylthio, alkylsulfinyl, alkylsulfonyl, alkenyl, cycloalkyl, cycloalkyloxy, cycloalkenyl, cycloalkenyloxy, polycyclyl, polycyclyloxy, heterocycloalkyl, aryl, aryloxy or heteroaryl, what has been said above is fully incorporated by reference. Radicals R¹ to R⁹ which in the above-mentioned formulae (IV) are bound to a carbon atom and have a heteroatom or a heteroatom-comprising group can also be bound directly via a heteroatom to the carbon atom.

If two adjacent radicals R¹ to R⁹ together with the ring atoms to which they are bound form at least one fused, saturated, unsaturated or aromatic ring or a ring system having from 1 to 30 carbon atoms, where the ring or the ring system may have from 1 to 5 nonadjacent heteroatoms or heteroatom-comprising groups and the ring or the ring system may be unsubstituted or substituted, these radicals may together as fused-on building blocks preferably be 1,3-propylene, 1,4-butylene, 1,5-pentylene, 2-oxa-1,3-propylene, 1-oxa-1,3-propylene, 2-oxa-1,3-propylene, 1-oxa-1,3-propenylene, 3-oxa-1,5-pentylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

The radical R in the compounds of the formulae IV.a to IV.v is preferably

unsubstituted C₁-C₁₈-alkyl such as methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl and 1-octadecyl;
C₁-C₁₈-alkyl substituted by one or more hydroxy, halogen, phenyl, cyano, C₁-C₆-alkoxycarbonyl and/or SO₃H radicals, especially hydroxy-C₁-C₁₅-alkyl such as 2-hydroxyethyl or 6-hydroxyhexyl; phenyl-C₁-C₁₈-alkyl such as benzyl, 3-phenylpropyl; cyano-C₁-C₁₈-alkyl such as 2-cyanoethyl; C₁-C₆-alkoxy-C₁-C₁₈-alkyl such as 2-(methoxycarbonyl)ethyl, 2-(ethoxycarbonyl)ethyl or 2-(n-butoxy-carbonyl)ethyl; fluoroalkyl such as trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl; sulfo-C₁-C₁₈-alkyl such as 3-sulfopropyl;
hydroxyethyloxyalkyl, radicals of oligoalkylene and polyalkylene glycols such as polyethylene glycols and polypropylene glycols and their oligomers having from 2 to 100 units and a hydrogen or a C₁-C₈-alkyl as end group, for example R^{A}O—(CHR^{B}—CH₂—O)ₙ—CHR^{B}—CH₂— where R^{A} and R^{B} is preferably hydrogen, methyl or ethyl and n is preferably from 0 to 3, in particular 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetra-oxamidecyl and 3,6,9,12-tetraoxatetradecyl; and
C₂-C₆-alkenyl such as vinyl or propenyl.

The radical R is particularly preferably linear C₁-C₁₈-alkyl such as methyl, ethyl, 1-propyl, 1-butyl, 1-pentyl, 1-hexyl, 1-heptyl, 1-octyl, 1-decyl, 1-dodecyl, 1-tetradecyl, 1-hexadecyl, 1-octadecyl, very particularly preferably methyl, ethyl, 1-butyl and 1-octyl, and also CH₃O—(CH₂CH₂O)ₙ—CH₂CH₂— and CH₃CH₂O—(CH₂CH₂O)ₘ—CH₂CH₂— where m is from 0 to 3.

Preference is given to the radicals R¹ to R⁹ in the compounds of the formulae IV.a to IV.v each being, independently of one another, H, halogen, hydroxy, alkoxy, alkylthio, carboxyl, —COOH, sulfonate, CN, NO₂, acyl, alkoxycarbonyl, NE¹E², where E¹ and E² are as defined above,

C₁-C₁₈-alkyl which is unsubstituted or substituted and/or may be interrupted by at least one heteroatom or heteroatom-comprising group,
C₂-C₁₈-alkenyl which is unsubstituted or substituted and/or may be interrupted by at least one heteroatom,
C₆-C₁₀-aryl which is unsubstituted or substituted,
C₅-C₁₂-cycloalkyl which is unsubstituted or substituted,
polycyclyl which is unsubstituted or substituted,
C₅-C₁₂-cycloalkenyl which is unsubstituted or substituted,
heterocycloalkyl having 5 or 6 ring atoms, where the ring has 1, 2 or 3 heteroatoms or heteroatom-comprising groups selected from among oxygen, nitrogen, sulfur and NR^{E} in addition to ring carbons, and is unsubstituted or substituted or
heteroaryl having 5 or 10 ring atoms, where the ring has 1, 2 or 3 heteroatoms or heteroatom-comprising groups selected from among oxygen, nitrogen, sulfur and NR^{E} in addition to ring carbons, and is unsubstituted or substituted.

Preference is likewise given to two adjacent radicals R¹ to R⁹ in the compounds of the formulae IV.a to IV.v together with the ring atoms to which they are bound also being able to form at least one fused, saturated, unsaturated or aromatic ring or a ring system having from 1 to 12 carbon atoms, where the ring or the ring system may have from 1 to 5 nonadjacent heteroatoms or heteroatom-comprising groups which are preferably selected from among oxygen, nitrogen, sulfur and NR^{E} and the ring or the ring system is unsubstituted or may be substituted by substituents which are preferably selected independently from among alkoxy, cycloalkyl, cycloalkoxy, polycyclyl, polycyclyloxy, heterocycloalkyl, aryl, aryloxy, arylthio, heteroaryl, halogen, hydroxy, SH, ═O, ═S, ═NR^{E}, COOH, carboxylate, —SO₃H, sulfonate, NE¹E², nitro and cyano, where E¹ and E² are each, independently of one another, hydrogen, alkyl, cycloalkyl, heterocycloalkyl, aryl or hetaryl.

When R¹ to R⁹ in the compounds of the formulae IV.a to IV.v are alkoxy, then R¹ to R⁹ are preferably methoxy or ethoxy or R^{A}O—(CH₂CH₂CH₂CH₂O)ₙ—CH₂CH₂CH₂CH₂O— where R^{A} and R^{B} are preferably hydrogen, methyl or ethyl and n is preferably from 0 to 3.

When R¹ to R⁹ in the compounds of the formulae IV.a to IV.v are acyl, then these are preferably selected from among formyl and C₁-C₄-alkylcarbonyl, in particular formyl or acetyl.

When R¹ to R⁹ in the compounds of the formulae IV.a to IV.v are C₁-C₁₈-alkyl, then these are preferably selected from among unsubstituted C₁-C₁₈-alkyl such as methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-9-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, 2-ethylhexyl, 2,4,4-trimethyl-pentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl;

C₁-C₁₈-haloalkyl, especially C₁-C₁₈-fluoroalkyl, for example trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylisopentyl, C₆F₁₃, C₈F₁₇, C₁₀F₂₁, C₁₂F₂₅, especially C₁-C₁₈-chloroalkyl such as chloromethyl, 2-chloroethyl, trichloromethyl, 1,1-dimethyl-2-chloroethyl;
amino-C₁-C₁₈-alkyl such as 2-aminoethyl, 2-aminopropyl, 3-aminopropyl, 4-aminobutyl, 6-aminohexyl;
C₁-C₆-alkylamino-C₁-C₁₈-alkyl such as 2-methylaminoethyl, 2-methylaminopropyl, 3-methylaminopropyl, 4-methylaminobutyl, 6-methylaminohexyl;
di(C₁-C₆-alkyl)-C₁-C₁₈-alkyl such as 2-dimethylaminoethyl, 2-dimethylaminopropyl, 3-dimethylaminopropyl, 4-dimethylaminobutyl, 6-dimethylaminohexyl;
cyano-C₁-C₁₈-alkyl such as 2-cyanoethyl, 2-cyanopropyl;
C₁-C₁₀-alkoxy-C₁-C₁₈-alkyl such as methoxymethyl, 2-methoxyethyl, 2-methoxypropyl, 3-methoxypropyl, 2-methoxyisopropyl, 4-methoxybutyl, 6-methoxyhexyl, 2-ethoxyethyl, 2-ethoxypropyl, 3-ethoxypropyl, 4-ethoxybutyl, 6-ethoxyhexyl, 2-isopropoxyethyl, 2-butoxyethyl, 2-butoxypropyl, 2-octyloxyethyl, 5-methoxy-3-oxapentyl, 8-methoxy-3,6-dioxaoctyl, 7-methoxy-4-oxaheptyl, 11-methoxy-4,8-dioxaundecyl, 9-methoxy-5-oxanonyl, 9-methoxy-5-oxanonyl, 14-methoxy-5,10-dioxatetradecyl, 5-ethoxy-3-oxapentyl, 8-ethoxy-3,6-dioxaoctyl, 7-ethoxy-4-oxaheptyl, 11-ethoxy-4,8-dioxaundecyl, 9-ethoxy-5-oxanonyl or 14-ethoxy-5,10-oxatetradecyl, 15-methoxy-4,8,12-trioxapentadecyl, 11-methoxy-3,6,9-trioxaundecyl, 11-ethoxy-3,6,9-trioxaundecyl, 15-ethoxy-4,8,12-trioxapentadecyl;
di(C₁-C₁₀-alkoxy-C₁-C₁₈-alkyl) such as diethoxymethyl or diethoxyethyl, C₁-C₆-alkoxycarbonyl-C₁-C₁₈-alkyl such as 2-(methoxycarbonyl)ethyl, 2-(ethoxycarbonyl)ethyl, 2-(n-butoxycarbonyl)ethyl;
di(C₁-C₆-alkoxycarbonyl)-C₁-C₁₈-alkyl such as 1,2-di(methoxycarbonyl)ethyl;
hydroxy-C₁-C₁₈-alkyl such as 2-hydroxyethyl, 2-hydroxypropyl, 3-hydroxypropyl, 4-hydroxybutyl, 6-hydroxyhexyl, 2-hydroxy-2,2-dimethylethyl, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-dioxatetradecyl;
C₁-C₁₂-alkylsulfanyl-C₁-C₁₈-alkyl such as butylthiomethyl, 2-dodecylthioethyl;
C₅-C₁₂-cycloalkyl-C₁-C₁₈-alkyl such as cyclopentylmethyl, 2-cyclopentylethyl, 3-cyclopentylpropyl, cyclohexylmethyl, 2-cyclohexylethyl, 3-cyclohexylpropyl, phenyl-C₁-C₁₈-alkyl, where the phenyl part of phenyl-C₁-C₁₈-alkyl is unsubstituted or monosubstituted, disubstituted, trisubstituted or tetrasubstituted by substituents selected independently from among C₁-C₆-alkyl, halogen, C₁-C₆-alkoxy and nitro, e.g. benzyl (phenylmethyl), 1-phenylethyl, 2-phenylethyl, 3-phenylpropyl, p-tolylmethyl, 1-(p-butylphenyl)ethyl, p-chlorobenzyl, 2,4-dichlorobenzyl, p-methoxybenzyl, m-ethoxybenzyl, phenyl-C(CH₃)₂—, 2,6-dimethylphenylmethyl;
diphenyl-C₁-C₁₈-alkyl such as diphenylmethyl (benzhydryl);
triphenyl-C₁-C₁₈-alkyl such as triphenylmethyl;
phenoxy-C₁-C₁₈-alkyl such as 2-phenoxyethyl, 2-phenoxypropyl, 3-phenoxypropyl, 4-phenoxybutyl, 6-phenoxyhexyl; and
phenylthio-C₁-C₁₈-alkyl such as 2-phenylthioethyl.

When R¹ to R⁹ in the compounds of the formulae IV.a to IV.v are C₂-C₁₈-alkenyl, then these are preferably selected from among C₂-C₆-alkenyl such as vinyl, 2-propenyl, 3-butenyl, cis-2-butenyl, trans-2-butenyl and C₂-C₁₈-alkenyl which may be partially or fully substituted by fluorine.

When R¹ to R⁹ in the compounds of the formulae IV.a to IV.v are C₆-C₁₀-aryl, then R¹ to R⁹ are preferably phenyl or naphthyl, where phenyl or naphthyl is unsubstituted or substituted by one, two, three or four substituents selected independently from among halogen, C₁-C₁₈-alkyl, C₁-C₆-alkoxy, C₁-C₆-alkylsulfanyl, C₁-C₆-alkoxy-C₁-C₆-alkyl, C₁-C₆-alkylcarbonyl, amino, C₁-C₆-alkylamino, di(C₁-C₆-dialkyl)amino and nitro, e.g. phenyl, methylphenyl (tolyl), dimethylphenyl (xylyl) such as 2,6-dimethylphenyl, trimethylphenyl such as 2,4,6-trimethylphenyl, ethylphenyl, diethylphenyl, isopropylphenyl, tert-butylphenyl, dodecylphenyl, chlorophenyl, dichlorophenyl, trichlorophenyl, fluorophenyl, difluorophenyl, trifluorophenyl, tetrafluorophenyl, pentafluorophenyl, 2,6-dichlorophenyl, 4-bromophenyl, methoxyphenyl, dimethoxy-phenyl, ethoxyphenyl, hexyloxyphenyl, 2,6-dimethoxyphenyl, 2-nitrophenyl, 4-nitrophenyl, 2,4-dinitrophenyl, 2,6-dinitrophenyl, 4-dimethylaminophenyl, 4-acetylphenyl, methoxyethylphenyl, ethoxymethylphenyl, methylthiophenyl, isopropylthiophenyl, tert-butylthiophenyl, α-naphthyl, β-naphthyl, methylnaphthyl, isopropylnaphthyl, chloronaphthyl, ethoxynaphthyl, or partially fluorinated phenyl or perfluorinated phenyl.

When R¹ to R⁹ in the compounds of the formulae IV.a to IV.v are C₅-C₁₂-cycloalkyl, then R¹ to R⁹ are preferably unsubstituted cycloalkyl such as cyclopentyl or cyclohexyl; C₅-C₁₂-cycloalkyl which is monosubstituted or disubstituted by substituents selected independently from among C₁-C₈-alkyl, C₁-C₆-alkoxy, C₁-C₆-alkylsulfanyl and chlorine, e.g. butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl; C₅-C₁₂-cycloalkyl which is completely or fully fluorinated.

When R¹ to R⁹ in the compounds of the formulae IV.a to IV.v are polycyclyl, then R¹ to R⁹ are preferably C₅-C₁₂-bicycloalkyl such as norbornyl or C₅-C₁₂-bicycloalkenyl such as norbornenyl.

When R¹ to R⁹ in the compounds of the formulae IV.a to IV.v are C₅-C₁₂-cycloalkenyl, then R¹ to R⁹ are preferably unsubstituted cycloalkenyl such as cyclopent-2-en-1-yl, cyclopent-3-en-1-yl, cyclohex-2-en-1-yl, cyclohex-1-en-1-yl, cyclohexa-2,5-dien-1-yl or partially or fully fluorinated cycloalkenyl.

When R¹ to R⁹ in the compounds of the formulae IV.a to IV.v are heterocycloalkyl having 5 or 6 ring atoms, then R¹ to R⁹ are preferably 1,3-dioxolan-2-yl, 1,3-dioxan-2-yl, 2-methyl-1,3-dioxolan-2-yl, 4-methyl-1,3-dioxolan-2-yl.

When R¹ to R⁹ in the compounds of the formulae IV.a to IV.v are heteroaryl, then R¹ to R⁹ are preferably furyl, thienyl, pyrryl, pyridyl, indolyl, benzoxazolyl, benzimidazolyl, benzthiazolyl. If it is substituted, hetaryl bears 1, 2 or 3 substituents selected independently from among C₁-C₆-alkyl, C₁-C₆-alkoxy and halogen, for example dimethylpyridyl, methylquinolyl, dimethylpyrryl, methoxyfuryl, dimethoxypyridyl or difluoropyridyl.

Particular preference is given to the radicals R¹ to R⁹ in the compounds of the formulae IV.a to IV.v each being, independently of one another, hydrogen; unbranched or branched C₁-C₁₈-alkyl which may be unsubstituted or substituted by one or more hydroxy, halogen, phenyl, cyano, C₁-C₆-alkoxycarbonyl and/or sulfo groups, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl, 1-octadecyl, 2-hydroxyethyl, benzyl, 3-phenylpropyl, 2-cyanoethyl, methoxycarbonylmethyl, ethoxycarbonylmethyl, n-butoxycarbonylmethyl, tert-butoxycarbonylmethyl, 2-(methoxycarbonyl)ethyl, 2-(ethoxycarbonyl)ethyl, 2-(n-butoxycarbonyl)ethyl, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl, 6-hydroxyhexyl and 3-sulfopropyl;

hydroxyethyloxyalkyl, radicals of oligoalkylene and polyalkylene glycols such as polyethylene glycols and polypropylene glycols and their oligomers having from 2 to 100 units and a hydrogen or a C₁-C₈-alkyl as end group, for example R^{A}O—(CHR^{B}—CH₂—O)ₙ—CHR^{B}—CH₂— or R^{A}O—(CH₂CH₂CH₂CH₂O)ₙ—CH₂CH₂CH₂CH₂O— where R^{A} and R^{B} are each preferably hydrogen, methyl or ethyl and n is preferably from 0 to 3, in particular 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl;
C₂-C₄-alkenyl such as vinyl and allyl; and
N,N-di-C₁-C₆-alkylamino such as N,N-dimethylamino and N,N-diethylamino.

Very particular preference is given to the radicals R¹ to R⁹ each being, independently of one another, hydrogen; C₁-C₁₈-alkyl such as methyl, ethyl, 1-butyl, 1-pentyl, 1-hexyl, 1-heptyl, 1-octyl; phenyl; 2-hydroxyethyl; 2-cyanoethyl; 2-(alkoxycarbonyl)ethyl such as 2-(methoxycarbonyl)ethyl, 2-(ethoxycarbonyl)ethyl or 2-(n-butoxycarbonyl)ethyl;

N,N—(C₁-C₄-dialkyl)amino such as N,N-dimethylamino or N,N-diethylamino; chlorine and radicals of oligoalkylene glycol such as CH₃O—(CH₂CH₂O)ₙ—CH₂CH₂— or CH₃CH₂O—(CH₂CH₂O)ₙ—CH₂CH₂— where n is from 0 to 3.

Preferred pyridinium ions are compounds of the formula IV.a in which one of the radicals R¹ to R⁵ is methyl, ethyl or chlorine and the remaining radicals R¹ to R⁵ are each H.

Further preferred pyridinium ions are compounds of the formula IV.a in which R³ is dimethylamino and the remaining radicals R¹, R², R⁴ and R⁵ are each H.

Further preferred pyridinium ions are compounds of the formula IV.a in which the radicals R¹ to R⁵ are each H.

Further preferred pyridinium ions are compounds of the formula IV.a in which R² is carboxy or carboxamide and the remaining radicals R¹, R², R⁴ and R⁵ are each H.

Further preferred pyridinium ions are compounds of the formula IV.a in which R¹ and R² or R² and R³ are together 1,4-buta-1,3-dienylene and the remaining radicals R¹, R², R⁴ and R⁵ are each H.

Particularly preferred pyridinium ions are pyridinium, 2-methylpyridinium, 2-ethylpyridinium, 5-ethyl-2-methylpyridinium and 2-methyl-3-ethylpyridinium and also 1-methylpyridinium, 1-ethylpyridinium, 1-(1-butyl)pyridinium, 1-(1-hexyl)pyridinium, 1-(1-octyl)pyridinium, 1-(1-hexyl)pyridinium, 1-(1-octyl)pyridinium, 1-(1-dodecyl)pyridinium, 1-(1-tetradecyl)pyridinium, t-(1-hexadecyl)pyridinium, 1,2-dimethyl-pyridinium, 1-ethyl-2-methylpyridinium, 1-(1-butyl)-2-methylpyridinium, 1-(1-hexyl)-2-methylpyridinium, 1-(1-octyl)-2-methyl-pyridinium, 1-(1-dodecyl)-2-methylpyridinium, 1-(1-tetradecyl)-2-methylpyridinium, 1-(1-hexadecyl)-2-methylpyridinium, 1-methyl-2-ethylpyridinium, 1,2-diethylpyridinium, 1-(1-butyl)-2-ethylpyridinium, 1-(1-hexyl)-2-ethylpyridinium, 1-(1-octyl)-2-ethyl-pyridinium, 1-(1-dodecyl)-2-ethylpyridinium, 9-(1-tetradecyl)-2-ethylpyridinium, 1-(1-hexadecyl)-2-ethylpyridinium, 1,2-dimethyl-5-ethylpyridinium, 1,5-diethyl-2-methyl-pyridinium, 1-(1-butyl)-2-methyl-3-ethylpyridinium, 1-(1-hexyl)-2-methyl-3-ethyl-pyridinium and 1-(1-octyl)-2-methyl-3-ethylpyridinium, 1-(1-dodecyl)-2-methyl-3-ethyl-pyridinium, 1-(1-tetradecyl)-2-methyl-3-ethylpyridinium and 1-(1-hexadecyl)-2-methyl-3-ethylpyridinium.

Preferred pyridazinium ions are compounds of the formula IV.b in which the radicals R¹ to R⁴ are each H or in which one of the radicals R¹ to R⁴ is methyl or ethyl and the remaining radicals R¹ to R⁴ are each H.

Preferred pyrimidinium ions are compounds of the formula IV.c in which R¹ is H, methyl or ethyl and R² to R⁴ are each, independently of one another, H or methyl or in which R¹ is H, methyl or ethyl, R² and R⁴ are each methyl and R³ is H.

Preferred pyrazinium ions are compounds of the formula IV.d in which R¹ is H, methyl or ethyl and R² to R⁴ are each, independently of one another, H or methyl or in which R¹ is H, methyl or ethyl and R² and R⁴ are each methyl and R³ is H or in which R¹ to R⁴ are each methyl or in which R¹ to R⁴ are each H.

Preferred imidazolium ions are compounds of the formula IV.e in which R¹ is H, methyl, ethyl, 1-propyl, 1-butyl, 1-pentyl, 1-hexyl, 1-octyl, 2-hydroxyethyl or 2-cyanoethyl and R² to R⁴ are each, independently of one another, H, methyl or ethyl.

Particularly preferred imidazolium ions of the formula IV.e are 1-methylimidazolium, 1-ethylimidazolium, 1-(1-propyl)imidazolium, 1-(1-allyl)imidazolium, 1-(1-butyl)imidazolium, 1-(1-octyl)imidazolium, 1-(1-dodecyl)imidazolium, 1-(1-tetradecyl)imidazolium, 1-(1-hexadecyl)imidazolium, 1,3-dimethylimidazolium, 1,3-diethylimidazolium, 1-ethyl-3-methylimidazolium, 1-(1-butyl)-3-methylimidazolium, 1-(1-butyl)-3-ethylimidazolium, 1-(1-hexyl)-3-methyl-imidazolium, 1-(1-hexyl)-3-ethylimidazolium, 1-(1-hexyl)-3-butylimidazolium, 1-(1-octyl)-3-methylimidazolium, 1-(1-octyl)-3-ethylimidazolium, 1-(1-octyl)-3-butylimidazolium, 1-(1-dodecyl)-3-methyl-imidazolium, 1-(1-dodecyl)-3-ethylimidazolium, 1-(1-dodecyl)-3-butylimidazolium, 1-(1-dodecyl)-3-octylimidazolium, 1-(1-tetradecyl)-3-methylimidazolium, 1-(1-tetradecyl)-3-ethylimidazolium, 1-(1-tetradecyl)-3-butylimidazolium, 1-(1-tetradecyl)-3-octylimi-dazolium, 1-(1-hexadecyl)-3-methylimidazolium, 1-(1-hexadecyl)-3-ethylimidazolium, 1-(1-hexadecyl)-3-butylimidazolium, 1-(1-hexadecyl)-3-octylimidazolium, 1,2-dimethyl-imidazolium, 1,2,3-trimethylimidazolium, 1-ethyl-2,3-dimethylimidazolium, 1-(1-butyl)-2,3-dimethylimidazolium, 1-(1-hexyl)-2,3-dimethylimidazolium, 1-(1-octyl)-2,3-di-methylimidazolium, 1,4-dimethylimidazolium, 1,3,4-trimethylimidazolium, 1,4-dimethyl-3-ethylimidazolium, 3-methylimidazolium, 3-ethylimidazolium, 3-n-propylimidazolium, 3-n-butylimidazolium, 1,4-dimethyl-3-octylimidazolium, 1,4,5-trimethylimidazolium, 1,3,4,5-tetramethylimidazolium, 1,4,5-trimethyl-3-ethylimidazolium, 1,4,5-trimethyl-3-butylimidazolium, 1,4,5-trimethyl-3-octylimidazolium, 1-prop-1-en-3-yl-3-methylimidazolium and 1-prop-1-en-3-yl-3-butylimidazolium.

Preferred pyrazolium ions are compounds of the formulae IV.f, IV.g and IV.g′ in which R¹ is H, methyl or ethyl and R² to R⁴ are each, independently of one another, H or methyl.

Further preferred pyrazolium ions are compounds of the formula IV.h in which R¹ to R⁴ are each, independently of one another, H or methyl.

Particularly preferred pyrazolium ions are 1,4-dimethylpyrazolium and 1,2,4-trimethyl-pyrazolium.

Preferred 1-pyrazolinium ions are compounds of the formula IV.i in which R¹ to R⁶ are each, independently of one another, H or methyl.

Preferred 2-pyrazolinium ions are compounds of the formulae IV.j and IV.j′ in which R¹ is H, methyl, ethyl or phenyl and R² to R⁶ are each, independently of one another, H or methyl.

Preferred 3-pyrazolinium ions are compounds of the formulae IV.k. and .IV.k′ in which R¹ and R² are each, independently of one another, H, methyl, ethyl or phenyl and R³ to R⁶ are each, independently of one another, H or methyl.

Preferred imidazolinium ions are compounds of the formula (IV.l) in which R¹ and R² are each, independently of one another, H, methyl, ethyl, 1-butyl or phenyl, R³ and R⁴ are each, independently of one another, H, methyl or ethyl and R⁵ and R⁶ are each, independently of one another, H or methyl.

Further preferred imidazolinium ions are compounds of the formulae IV.m and IV.m′ in which R¹ and R² are each, independently of one another, H, methyl or ethyl and R³ to R⁶ are each, independently of one another, H or methyl.

Further preferred imidazolinium ions are compounds of the formulae IV.n and IV.n′ in which R¹ to R³ are each, independently of one another, H, methyl or ethyl and R⁴ to R⁶ are each, independently of one another, H or methyl.

Preferred thiazolium ions are compounds of the formulae IV.o and IV.o′ in which R¹ is H, methyl, ethyl or phenyl and R² and R³ are each, independently of one another, H or methyl.

Preferred oxazolium ions are compounds of the formula IV.p in which R¹ is H, methyl, ethyl or phenyl and R² and R³ are each, independently of one another, H or methyl.

Preferred 1,2,4-triazolium ions are compounds of the formulae IV.q, IV.q′ and IV.q″ in which R¹ and R² are each, independently of one another, H, methyl, ethyl or phenyl and R³ is H, methyl or phenyl.

Preferred 1,2,3-triazolium ions are compounds of the formulae IV.r, IV.r′ and IV.r″ in which R¹ is H, methyl or ethyl, R² and R³ are each, independently of one another, H or methyl or R² and R³ are together 1,4-buta-1,3-dienylene.

Preferred pyrrolidinium ions are compounds of the formula IV.s in which R¹ is H, methyl, ethyl or phenyl and R² to R⁹ are each, independently of one another, H or methyl.

Preferred imidazolidinium ions are compounds of the formula IV.t in which R¹ and R⁴ are each, independently of one another, H, methyl, ethyl or phenyl and R², R³ and R⁵ to R⁸ are each, independently of one another, H or methyl.

Preferred diazabicycloalkenium ions of the formulae IV.u and IV.v are selected from among cationic derivatives of 1,5-diazabicyclo[4.3.0]non-5-ene (DBN) and 1,8-diaza-bicyclo[5.4.0]undec-7-ene (DBU).

In a particularly preferred embodiment of the process of the invention, the hydrolysis stability of an anionic liquid IL which has at least one cation selected from among the abovementioned imidazolium ions and the abovementioned pyrazolium ions is improved. The cation of the anionic liquid is very particularly preferably selected from among the abovementioned imidazolium ions. As regards preferred imidazolium ions and pyrazolium ions, what has been said above is fully incorporated by reference.

In the process of the invention, the anion [Y]ⁿ⁻ of the ionic liquid IL is preferably selected from among compounds of the formulae (R^{a}O)SO₃⁻, (R^{a})SO₃⁻, (R^{a}O)SO₂⁻, (R^{a}O)PO₃²⁻, (R^{a}O)(R^{b}O)PO₂⁻, (R^{a}O)(R^{b})PO₂⁻, (R^{a}O)PO₂²⁻, (R^{a}O)(R^{b}O)PO⁻, (R^{a}O)(R^{b})PO⁻, (R^{a}O)BO₂²⁻, (R^{a}O)(R^{b}O)BO⁻, (R^{a}O)(R^{b})BO⁻, (R^{a}O)(R^{b}O)(R^{c}O)(R^{d}O)B⁻, (R^{a}O)CO₂⁻, (R^{a}O)SiO₃³⁻, (R^{a}O)(R^{b}O)SiO₂²⁻, (R^{a}O)(R^{b})SiO₂²⁻, (R^{a}O)(R^{b}O)(R^{d}O)SiO⁻, (R^{a}O)(R^{b}O)(R^{c})SiO⁻ and (R^{a}O)(R^{b})(R^{c})SiO⁻,

where the radicals R^{a}, R^{b}, R^{c} and R^{d} are each, independently of one another, H, alkyl, preferably C₁-C₃₀-alkyl, particularly preferably C₁-C₁₈-alkyl, which is unsubstituted or substituted and/or may be interrupted by at least one heteroatom or heteroatom-comprising group,
aryl, preferably C₆-C₁₄-aryl, particularly preferably C₆-C₁₀-aryl, which is unsubstituted or substituted,
cycloalkyl, preferably C₅-C₁₂-cycloalkyl, which is unsubstituted or substituted, heterocycloalkyl, preferably heterocycloalkyl having 5 or 6 ring atoms, where the ring has 1, 2 or 3 heteroatoms or heteroatom-comprising groups in addition to ring carbons, which is unsubstituted or substituted,
heteroaryl, preferably heteroaryl having from 5 to 10 ring atoms, where the ring has 1, 2 or 3 heteroatoms or heteroatom-comprising groups selected from among oxygen, nitrogen, sulfur and NR^{E} in addition to ring carbons, which is unsubstituted or substituted,
where, in anions which have a plurality of radicals R^{a} to R^{d} two of these radicals together with the part of the anion to which they are bound can also form at least one saturated, unsaturated or aromatic ring or ring system having from 1 to 12 carbon atoms, where the ring or ring system can have from 1 to 5 nonadjacent heteroatoms or heteroatom-comprising groups which are preferably selected from among oxygen, nitrogen, sulfur and NR^{E} and the ring or ring system is unsubstituted or may be substituted.

As regards suitable and preferred C₁-C₃₀-alkyls, in particular C₁-C₁₈-alkyls, C₆-C₁₄-aryls, in particular C₈-C₁₀-aryls, C₅-C₁₂-cycloalkyls, heterocycloalkyls having 5 or 6 ring atoms and heteroaryls having 5 or 6 ring atoms, what has been said above is incorporated by reference. As regards suitable and preferred substituents on C₁-C₃₀-alkyl, especially C₁-C₁₃-alkyl, C₆-C₁₂-aryl, C₅-C₁₂-cycloalkyl, heterocycloalkyl having 5 or 6 ring atoms and heteroaryl having 5 or 6 ring atoms, what has been said above in respect of substituents is likewise incorporated by reference.

When at least one of the radicals R^{a} to R^{d} is optionally substituted C₁-C₁₈-alkyl, then it is preferably methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, hetadecyl, octadecyl, 1,1-dimethylpropyl, 1,1-dimethylbutyl, 1,1,3,3-tetramethylbutyl, benzyl, 1-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonyl-propyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethyl-aminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl.

When at least one of the radicals R^{a} to R^{d} is C₁-C₁₆-alkyl interrupted by one or more nonadjacent heteroatoms or heteroatom-comprising groups, then it is preferably 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxa-pentadecyl, 9-hydroxy-5-oxanonyl, 14-hydroxy-5,10-oxatetradecyl, 5-methoxy-3-oxa-pentyl, 8-methoxy-3,6-dioxaoctyl, 11-methoxy-3,6,9-trioxaundecyl, 7-methoxy-4-oxa-heptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

If two radicals R^{a} to R^{d} form a ring, these radicals can together form as fused-on building block, for example, 1,3-propylene, 1,4-butylene, 2-oxa-1,3-propylene, 1-oxa-1,3-propylene, 2-oxa-1,3-propenylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

The number of nonadjacent heteroatoms or heteroatom-comprising groups of the radicals R^{a} to R^{d} is in principle not critical and is generally restricted only by the size of the respective radical or cyclic building block. In general, there will be no more than 5 in the respective radical, preferably no more than 4 or very particularly preferably no more than 3. Furthermore, there is generally at least one carbon atom, preferably at least two carbon atoms, between any two heteroatoms.

Substituted and unsubstituted imino groups can be, for example, imino, methylimino, isopropylimino, n-butylimino or tert-butylimino.

Preferred functional groups of the radicals R^{a} to R^{d} are carboxy, carboxamide, hydroxyl, di(C₁-C₄-alkyl)amino, C₁-C₄-alkyloxycarbonyl, cyano or C₁-C₄-alkoxy. Radicals R^{e} to R^{f} which are not alkyl may additionally be monosubstituted or polysubstituted by C₁-C₄-alkyl, preferably methyl, ethyl, propyl, isopropyl, n-butyl, sec-butyl or tert-butyl.

When at least one of the radicals R^{a} to R^{d} is optionally substituted C₆-C₁₂-aryl, then it is preferably phenyl, methylphenyl (tolyl), xylyl, α-naphthyl, β-naphthyl, chlorophenyl, dichlorophenyl, trichlorophenyl, difluorophenyl, dimethylphenyl, trimethylphenyl, ethyl-phenyl, diethylphenyl, isopropylphenyl, tert-butylphenyl, dodecylphenyl, methoxy-phenyl, dimethoxyphenyl, ethoxyphenyl, hexyloxyphenyl, methylnaphthyl, isopropyl-naphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl.

When at least one of the radicals R^{a} to R^{e} is optionally substituted C₅-C₁₂-cycloalkyl, then it is preferably cyclopentyl, cyclohexyl, cyclooctyl, cyclododecyl, methylcyclo-pentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbornyl or norbornenyl.

When at least one of the radicals R^{a} to R^{e} is an optionally substituted five- or six-membered heterocycle, then it is preferably furyl, thienyl, pyryl, pyridyl, indolyl, benzoxazolyl, dioxolyl, dioxyl, benzimidazolyl, benzthiazolyl, dimethylpyridyl, methylquinolyl, dimethylpyrryl, methoxyfuryl, dimethoxypyridyl, difluoropyridyl, methylthiophenyl, isopropylthiophenyl or tert-butylthiophenyl.

When, in the case of anions having a plurality of radicals R^{a} to R^{e}, two of these radicals together with the part of the anion to which they are bound can form at least one saturated, unsaturated or aromatic ring or a ring system which has from 1 to 12 carbon atoms and may have from 1 to 5 nonadjacent heteroatoms or heteroatom-comprising groups which are preferably selected from among oxygen, nitrogen, sulfur and NR^{E}, then the ring or the ring system is unsubstituted or bears 1, 2, 3, 4, 5 or more than 5 substituents. The substituents are preferably selected independently from among alkyl, alkoxy, alkylsulfanyl, cycloalkyl, cycloalkoxy, polycyclyl, heterocycloalkyl, aryl, aryloxy, arylthio and heteroaryl.

The abovementioned anions or anion equivalents in ionic liquids are generally at least partly subject to hydrolysis.

In a particularly preferred embodiment of the process, the anion [Y]ⁿ⁻ of the ionic liquid (IL) is selected from among compounds of the formulae (R^{a}O)SO₃⁻, (R^{a})SO₃⁻, (R^{a}O)PO₃²⁻ and (R^{a}O)(R^{b}O)PO₂⁻ where R^{a} and R^{b} are each, independently of one another, alkyl, cycloalkyl or aryl, especially unsubstituted C₁-C₄-alkyl. The anion [Y]ⁿ⁻ of the ionic liquid (IL) is very particularly preferably selected from among compounds of the formula (R^{a}O)SO₃⁻, especially mono-C₁-C₄-alkylsulfates, for example ethylsulfate.

The anions mentioned as particularly preferred in ionic liquids are particularly subject to hydrolysis. In particular, the sulfuric acid obtained by hydrolysis of compounds of the formula (R^{a}O)SO₃⁻ can, due to its corrosive propertives, have a damaging effect on chemical compounds, chemical reactions and apparatuses with which an ionic liquid comprising this is brought into contact.

In a first embodiment of the process of the invention, tertiary amines or mixtures of tertiary amines are used for increasing the hydrolysis stability of the ionic liquid (IL).

Suitable tertiary amines are compounds of the formula NR¹R²R³, where R¹, R² and R³ have one of the meanings other than H given above for R¹ to R⁹.

In a specific embodiment, the tertiary amines used according to the invention are selected from among compounds of the formula NR¹R²R³, where R¹, R² and R³ are each, independently of one another, optionally substituted C₁-C₃₀-alkyl, C₃-C₈-cycloalkyl or aryl, where C₁-C₃₀-alkyl can also, as defined above, be interrupted by one or more nonadjacent heteroatoms or heteroatom-comprising groups.

Examples of suitable unsubstituted tertiary amines are triethylamine, diethyl-n-propylamine, diethylisopropylamine, diethyl-n-butylamine, diethyl-tert-butylamine, diethyl-n-pentylamine, diethylhexylamine, diethylcyclohexylamine, diethyloctylamine, diethyl-(2-ethylhexyl)amine, diethyldodecylamine tri-n-propylamine, di-n-propylethylamine, di-n-propylbutylamine, di-n-propyl-n-pentylamine, di-n-propylhexylamine, di-n-propylcyclohexylamine, di-n-propyloctylamine, di-n-propyl(2-ethylhexyl)amine, di-n-propyldodecylamine, triisopropylamine, diisopropyl-ethylamine, diisopropyl-n-propylamine, diisopropylbutylamine, diisopropylpentylamine, diisopropylhexylamine, diisopropylcyclohexylamine, diisopropyloctylamine, diisopropyl-(2-ethylhexyl)amine, diisopropyldodecylamine, tri-n-butylamine, di-n-butylethylamine, di-n-butyl-n-propylamine, di-n-butyl-n-pentylamine, di-n-butylhexylamine, di-n-butylcyclohexylamine, di-n-butyloctylamine, di-n-butyl-(2-ethylhexyl)amine, di-n-butyldodecylamine, N-benzyl-N-ethylaniline, N-benzyl-N-n-propylaniline, N-benzyl-N-isopropylaniline, N-benzyl-N-n-butylaniline, N,N-dimethyl-p-toluidine, N,N-diethyl-p-toluidine, N,N-di-n-butyl-p-toluidine, diethylbenzylamine, di-n-propylbenzylamine, di-n-butylbenzylamine, diethylphenylamine, di-n-propylphenylamine and di-n-butylphenylamine.

Examples of suitable substituted tertiary amines are tri(2-hydroxyethyl)amine, di(2-hydroxyethyl)-n-propylamine, di(2-hydroxyethyl)isopropylamine, di(2-hydroxyethyl)-n-butylamine, di(2-hydroxyethyl)-tert-butylamine, di(2-hydroxyethyl)-n-pentylamine, di(2-hydroxyethyl)hexylamine, di(2-hydroxyethyl)cyclohexylamine, di(2-hydroxyethyl)octylamine, di(2-hydroxyethyl)(2-ethylhexyl)amine, di(2-hydroxyethyl)nonylamine, di(2-hydroxyethyl)decylamine, di(2-hydroxyethyl)dodecylamine, di-n-propyl(2-hydroxyethyl)amine, diisopropyl-(2-hydroxyethyl)amine, di-n-butyl(2-hydroxyethyl)amine, N-benzyl-N-(2-hydroxyethyl)aniline, N,N-di(2-hydroxyethyl)-p-toluidine, di(2-hydroxyethyl)-benzylamine, di(2-hydroxyethyl)phenylamine and their derivatives which can be obtained by alkoxylation, especially by ethoxylation, of the 2-hydroxyethyl groups. The abovementioned alkoxylated derivatives of the tertiary amines are usually present as mixtures and have on average from 1 to 50, preferably from 1 to 20 and particularly preferably from 2 to 10, alkylene oxide units per 2-hydroxyethyl group. Preferred substituted tertiary amines are di(2-hydroxyethyl)(C₁-C₁₂-alkyl)amines and tri(2-hydroxyethyl)amine and their alkoxylated derivatives. A particularly preferred substituted tertiary amine is tri(2-hydroxyethyl)amine.

Mixtures of tertiary amines of the formula NR¹R²R³, where the meaning of at least one of the radicals R¹, R² or R³ is derived from a mixture of straight-chain and branched C₁-C₃₀-alkyl, especially C₈-C₁₈-alkyl, and C₁-C₃₀-alkenyl, especially C₈-C₁₈-alkenyl, as can be obtained from natural or synthetic fatty acids and fatty alcohols and from oxo alcohols, are likewise suitable. These include, for example, mixtures of n-octyl, n-nonyl, n-decyl, n-undecyl, n-dodecyl, n-tridecyl, myristyl, pentadecyl, palmityl (=cetyl), heptadecyl, octadecyl, nonadecyl, arachinyl (arachidyl), behenyl, octenyl, nonenyl, decenyl, undecenyl, dodecenyl, tridecenyl, tetradecenyl, pentadecenyl, hexadecenyl, heptadecenyl, octadecenyl, especially oleyl, nonadecenyl, linolyl, linolenyl or eleostearyl.

Preference is given to mixtures of tertiary amines of the formula NR¹R²R³ in which at least one of the radicals R¹, R² or R³ is a mixture of straight-chain and branched C₁-C₃₀-alkyl and C₁-C₃₀-alkenyl and at least one further, especially two, of the radicals R¹, R² or R³ is 2-hydroxyethyl or alkoxylated 2-hydroxyethyl.

In a further specific embodiment, the tertiary amines used according to the invention are selected from among compounds of the formula NR¹R²R³ in which R¹ together with R² and together with the nitrogen atom to which they are bound form a five- or six-membered heterocycle, with R³ having one of the meanings given above or together with an adjacent substituent of the heterocycle can form the single bond part of a chemical double bond.

Preference is given to heterocyclic tertiary amines NR¹R²R³ in which at least one ring carbon adjacent to a ring nitrogen has a substituent other than H, in particular a C₁-C₄-alkyl substituent.

Examples of suitable heterocyclic tertiary amines NR¹R²R³ are pyridine compounds, pyridazine compounds, pyrimidine compounds, pyrazine compounds, imidazole compounds, pyrazole compounds, 1,2,4-triazole compounds or 1,2,4-triazole compounds, in particular those which have at least one substituent in the position adjacent to a ring nitrogen.

Suitable pyridine compounds are, for example, 2-methylpyridine, 2-ethylpyridine, 2,3-dimethylpyridine, 2,4-dimethylpyridine, 2,5-dimethylpyridine, 2,6-dimethylpyridine, 5-ethyl-2-methylpyridine and 2-methyl-3-ethylpyridine.

Suitable pyridazine compounds are, for example, 3-methylpyridazine, 3-ethylpyridazine, 3,4-dimethylpyridazine, 3,5-dimethylpyridazine, 3,6-dimethylpyridazine.

Suitable pyrimidine compounds are, for example, 2-methylpyrimidine, 4-methylpyrimidine, 2,4-dimethylpyrimidine, 2,5-dimethylpyrimidine, 4,5-dimethylpyrimidine, 4,6-dimethylpyrimidine, 2,4,5-trimethylpyrimidine, 2,4,6-trimethylpyrimidine, 2-ethylpyrimidine, 2-ethyl-4-methylpyrimidine, 2-ethyl-5-methylpyrimidine, 2-ethyl-4,5-dimethylpyrimidine and 2-ethyl-4,6-dimethylpyrimidine.

Suitable pyrazine compounds are, for example, 2-methylpyrazine, 2,3-dimethylpyrazine, 2,5-dimethylpyrazine, 2,6-dimethylpyrazine, 2,3,5-trimethylpyrazine, 2,3,6-trimethylpyrazine, 2,3,5,6-tetramethylpyrazine, 2-ethylpyrazine, 2-ethyl-3-methylpyrazine, 2-ethyl-5-methylpyrazine, 2-ethyl-6-methylpyrazine, 2-ethyl-3,5-dimethylpyrazine, 2-ethyl-3,6-dimethylpyrazine and 2,3,5,6-tetramethylpyrazine.

Suitable imidazole compounds are, for example, 1,2-dimethylimidazole, 1-ethyl-2-methylimidazole, 1-n-propyl-2-methylimidazole, 1-isopropyl-2-methylimidazole, 1-n-butyl-2-methylimidazole, 1-sec-butyl-2-methylimidazole, 1-tert-butyl-2-methyl-imidazole, 1-(2-hydroxyethyl)-2-methylimidazole, 1,4-dimethylimidazole, 1-ethyl-4-methylimidazole, 1-n-propyl-4-methylimidazole, 1-isopropyl-4-methylimidazole, 1-n-butyl-4-methylimidazole, 1-sec-butyl-4-methylimidazole, 1-tert-butyl-4-methylimidazole, 1-(2-hydroxyethyl)-4-methylimidazole, 1,2,4-trimethylimidazole, 1,2,5-trimethyl-imidazole, 1,4,5-trimethylimidazole and 1,2,4,5-tetramethylimidazole.

Suitable pyrazole compounds are, for example, 1,3-dimethylpyrazole, 1-ethyl-3-methyl-pyrazole, 1-n-propyl-3-methylpyrazole, 1-isopropyl-3-methylpyrazole, 1-n-butyl-3-methylpyrazole, 1-sec-butyl-3-methylpyrazole, 1-tert-butyl-3-methylpyrazole, 1-(2-hydroxyethyl)-3-methylpyrazole, 1,5-dimethylpyrazole, 1-ethyl-5-methylpyrazole, 1-n-propyl-5-methylpyrazole, 1-isopropyl-5-methylpyrazole, 1-n-butyl-5-methylpyrazole, 1-sec-butyl-5-methylpyrazole, 1-tert-butyl-5-methylpyrazole, 1-(2-hydroxyethyl)-5-methylpyrazole, 1,3,4-trimethylpyrazole, 1,3,5-trimethylpyrazole, 1,4,5-trimethylpyrazole and 1,3,4,5-tetramethylpyrazole.

Suitable 1,2,4-triazole compounds are, for example, 1,3-dimethyl-1,2,4-triazole, 1-ethyl-3-methyl-1,2,4-triazole, 1-n-propyl-3-methyl-1,2,4-triazole, 1-isopropyl-3-methyl-1,2,4-triazole, 1-n-butyl-3-methyl-1,2,4-triazole, 1-sec-butyl-3-methyl-1,2,4-triazole, 1-tert-butyl-3-methyl-1,2,4-triazole, 1-(2-hydroxyethyl)-3-methyl-1,2,4-triazole, 1,5-dimethyl-1,2,4-triazole, 1-ethyl-5-methyl-1,2,4-triazole, 1-n-propyl-5-methyl-1,2,4-triazole, 1-iso-propyl-5-methyl-1,2,4-triazole, 1-n-butyl-5-methyl-1,2,4-triazole, 1-sec-butyl-5-methyl-1,2,4-triazole, 1-tert-butyl-5-methyl-1,2,4-triazole, 1-(2-hydroxyethyl)-5-methyl-1,2,4-triazole and 1,3,5-trimethyl-1,2,4-triazole.

Suitable 1,2,3-triazole compounds are, for example, 1,4-dimethyl-1,2,3-triazole, 1-ethyl-4-methyl-1,2,3-triazole, 1-n-propyl-4-methyl-1,2,3-triazole, 1-isopropyl-4-methyl-1,2,3-triazole, 1-n-butyl-4-methyl-1,2,3-triazole, 1-sec-butyl-4-methyl-1,2,3-triazole, 1-tert-butyl-4-methyl-1,2,3-triazole, 1-(2-hydroxyethyl)-4-methyl-1,2,3-triazole, 1,5-dimethyl-1,2,3-triazole, 1-ethyl-5-methyl-1,2,3-triazole, 1-n-propyl-5-methyl-1,2,3-triazole, 1-isopropyl-5-methyl-1,2,3-triazole, 1-n-butyl-5-methyl-1,2,3-triazole, 1-sec-butyl-5-methyl-1,2,3-triazole, 1-tert-butyl-5-methyl-1,2,3-triazole, 1-(2-hydroxyethyl)-5-methyl-1,2,3-triazole and 1,4,5-trimethyl-1,2,3-triazole.

Further suitable are 1,5-diazabicyclo[4.3.0]non-5-ene (DBN) and 1,8-diazabicyclo-[5.4.0]-undec-7-ene (DBU).

The heterocyclic tertiary amines of the formula NR¹R²R³ used according to the invention are preferably selected from among the abovementioned imidazole and pyrazole compounds. The heterocyclic tertiary amine is particularly preferably 1,2-dimethylimidazole.

In a further embodiment, various quaternary ammonium compounds or mixtures of quaternary ammonium compounds are used to increase the hydrolysis stability of the ionic liquid (IL).

Suitable quaternary ammonium compounds can, for example, be prepared by quaternization of the abovementioned tertiary amines NR¹R²R³ to give compounds of the formula [NR¹R²R³R]⁺(1/n)*[Y′]ⁿ⁻, where R has one of the meanings given in respect of the ionic liquids and (1/n)*[Y′]ⁿ⁻ is one anion equivalent. R in the quaternary ammonium compounds of the formula [NR¹R²R³R]⁺(1/n)*[Y′]ⁿ⁻ is preferably C₁-C₄-alkyl, particularly preferably methyl. The meaning of the anion equivalent (1/n)*[Y′]ⁿ⁻ is usually determined by the quaternization method selected, but can optionally be varied by means of anion exchange. In a specific embodiment, [Y′]ⁿ⁻ has one of the meanings given above for [Y]ⁿ⁻.

Suitable methods of quaternizing tertiary amines are known to those skilled in the art. One suitable method which may be mentioned here is, in particular, the reaction of tertiary amines of the formula NR¹R²R³ with C₁-C₄-alkyl halides such as methyl iodide or with di-C₁-C₄-alkyl sulfates such as dimethyl sulfate or diethyl sulfate.

Preferred quaternary ammonium compounds are (C₁-C₄-alkyl)(C₁-C₁₈-alkyl)-di(2-hydroxyethyl)ammonium compounds and (C₁-C₄-alkyl)tri(2-hydroxyethyl)-ammonium compounds and also their alkoxylated derivatives, in particular those which have a C₁-C₄-alkylsulfate anion as counterion. Particularly preferred quaternary ammonium compounds are methyltri(2-hydroxyethyl)ammonium compounds, in particular methylsulfates or ethylsulfates thereof, and also the alkoxylated derivatives of methyltri(2-hydroxyethyl)ammonium compounds.

Mixtures of quaternary ammonium compounds in which the meaning of at least one of the radicals R¹, R² or R³ is derived from a mixture of straight-chain and branched C₁-C₃₀-alkyl, especially C₈-C₁₈-alkyl, and C₁-C₃₀-alkenyl, especially C₈-C₁₈-alkenyl, as can be obtained from natural or synthetic fatty acids and fatty alcohols and also from oxo alcohols are also preferred. These include, for example, mixtures of n-octyl, n-nonyl, n-decyl, n-undecyl, n-dodecyl, n-tridecyl, myristyl, pentadecyl, palmityl (=cetyl), heptadecyl, octadecyl, nonadecyl, arachinyl (arachidyl), behenyl, octenyl, nonenyl, decenyl, undecenyl, dodecenyl, tridecenyl, tetradecenyl, pentadecenyl, hexadecenyl, heptadecenyl, octadecenyl, especially oleyl, nonadecenyl, linolyl, linolenyl or eleostearyl.

Particular preference is given to mixtures of quaternary ammonium compounds in which at least one of the radicals R¹, R² or R³ is a mixture of straight-chain and branched C₁-C₃₀-alkyl and C₁-C₃₀-alkenyl and at least one further, especially two, of the radicals R¹, R² or R³ is 2-hydroxyethyl or alkoxylated 2-hydroxyethyl. Such mixtures are commercially available, for example, under the trade name Ammoeng™ 100 (Solvent Solution).

The tertiary amines and/or quaternary ammonium compounds used according to the invention are preferably added in an amount of from 0.01 to 50% by weight, preferably in an amount of from 0.05 to 30% by weight and particularly preferably from 0.1 to 20% by weight, in each case based on the total weight of the ionic liquid IL.

The ionic liquids IL used according to the invention and the tertiary amines and/or quaternary ammonium compounds used according to the invention are advantageously completely miscible with one another, i.e. addition of the tertiary amine and/or the quaternary ammonium compound to an ionic liquid IL forms a homogeneous liquid composition.

The present invention is illustrated below by means of nonlimiting examples.

### EXAMPLES

### 1. Hydrolysis of Ionic Liquids as a Function of the Additive

A mixture of 1-ethyl-3-methylimidazolium ethylsulfate (EMIM-EtSO₄), 2% by weight of water and from 0.1 to 16% by weight of an additive (in each case based on the weight of EMIM-EtSO₄) was stirred at 150° C. for one hour. A sample was subsequently taken and examined by ¹H-NMR spectroscopy. The molar ratio of ethanol (hydrolysis product) to EMIM-EtSO₄ was determined from this spectrum by integration. The percentage hydrolysis of the ionic liquid (IL) used was determined from this ratio. The experiment was repeated three times for each additive. Table 1 shows the averaged results of these experiments.

TABLE 1

| | | | Amount | |
|---|---|---|---|---|
| | | | [% by | Hydrolysis |
| Ex. | IL | Additive | weight] | [%] |
| 1.1* | EMIM-EtSO₄ | — | — | 4.0 |
| 1.2 | EMIM-EtSO₄ | Triethanolamine | 0.5 | 0.0 |
| 1.3 | EMIM-EtSO₄ | 1,2-Dimethyl- | 0.5 | 0.0 |
| | | imidazole | | |
| 1.4 | EMIM-EtSO₄ | Siligen APE^{[1]} | 0.5 | 0.0 |
| 1.5 (CE)** | EMIM-EtSO₄ | P₄O₁₀ | 0.5 | 19.9 |
| 1.6 (CE)** | EMIM-EtSO₄ | Benzotriazole | 16 | 9.6 |
| 1.7*** | EMIM-EtSO₄ | Golpanol^{[2]} | 0.1 | 2.1 |
| 1.8 | EMIM-EtSO₄ | Siligen APE^{[1]} + | 16 + 0.1 | 0.0 |
| | | Golpanol^{[2]} | | |

*Reference measurement for Examples 1.2 to 1.4
**Comparative Examples (not according to the invention)
***Reference measurement for Example 1.8
^{[1]}Siligen APE^{(TM)} = tri(2-hydroxyethyl)methylammonium methylsulfate
^{[2]}Golpanol^{(TM)} = 2-butyne-1,4-diol

### 2. Stabilization of Ionic Liquids

A mixture of 1-ethyl-3-methylimidazolium ethylsulfate (EMIM-EtSO₄), 10% by weight of water and 16% by weight of an additive (in each case based on the weight of EMIM-EtSO₄) were stirred at 90° C. for one hour. Samples were taken at the times shown in Table 2 and the acid number and the pH (after addition of 10% of water to the sample taken) were determined. The results are shown in Table 2. As a result of the addition of the quaternary ammonium compounds used according to the invention, the ionic liquid is stabilized at an approximately neutral pH.

TABLE 2

| | | | | Acid number |
|---|---|---|---|---|
| Ex. | Additive | Time [d] | pH | [mg(KOH)/g] |
| 2.1* | — | 0 | 7.8 | — |
| 2.2 | Siligen APE ^{[1]} | 1 | 8 | 34 |
| 2.3 | Siligen APE ^{[1]} | 2 | 7.1 | 33.8 |
| 2.4 | Siligen APE ^{[1]} | 4 | 6.9 | 34.2 |
| 2.5 | Siligen APE ^{[1]} | 7 | 6.9 | 34 |
| 2.6 | Ammoeng ^{[3]} | 1 | 7.6 | 1.6 |
| 2.7 | Ammoeng ^{[3]} | 2 | 7.4 | 2 |
| 2.8 | Ammoeng ^{[3]} | 4 | 7.1 | 1.5 |
| 2.9 | Ammoeng ^{[3]} | 7 | 7.1 | 1.2 |

*: Reference measurement for Examples 2.1 to 2.9
^{[1]}: Siligen APE ⁽™⁾ = tri(2-hydroxyethyl)methylammonium methylsulfate

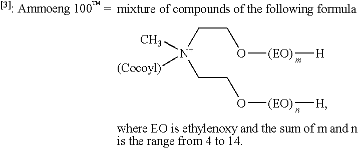

1. A method for improving the hydrolysis stability of an ionic liquid (IL), comprising adding at least one tertiary amine and/or one quaternary ammonium compound different from the ionic liquid (IL) to the ionic liquid (IL).

2. The method according to claim 1, wherein the ionic liquid (IL) is at least one ionic liquid selected from the group consisting of salts represented by formula (I)
[A]⁺(1/n)*[Y]ⁿ⁻ (I),
wherein [A]⁺ is a quaternary ammonium cation and (1/n)*[Y]ⁿ⁻ is an anion equivalent of an anion bearing n charges.

3. The method according to claim 2, wherein the quaternary ammonium cation [A]⁺ of the at least one ionic liquid has a molar mass of less than 1000 g/mol.

4. The method according to either claim 2, wherein the quaternary ammonium cation [A]⁺ comprises a heterocyclic cation.

5. The method according to claim 2, wherein the quaternary ammonium cation [A]⁺ is at least one selected from the group consisting of a pyridinium ion, pyridazinium ion, pyrimidinium ion, pyrazinium ion, imidazolium ion, pyrazolium ion, thiazolium ion, oxazolium ion, 1,2,4-triazolium ion, 1,2,3-triazolium ion, pyrrolidinium ion, imidazolidinium ion, and a diazabicycloalkenium ion.

6. The method according to claim 5, wherein the quaternary ammonium cation [A]⁺ is an imidazolium ion or a pyrazolium ion.

7. The method according to claim 6, wherein the quaternary ammonium cation [A]⁺ is an imidazolium ion.

8. The method according to claim 2, wherein an anion [Y]ⁿ⁻ of the at least one ionic liquid (IL) is selected from the group consisting of compounds represented by formulae (R^{a}O)SO₃⁻, (R^{a})SO₃⁻, (R^{a}O)SO₂⁻, (R^{a}O)PO₃²⁻, (R^{a}O)(R^{b}O)PO₂⁻, (R^{a}O)(R^{b})PO₂⁻, (R^{a}O)PO₂²⁻, (R^{a}O)(R^{b}O)PO⁻, (R^{a}O)(R^{b})PO⁻, (R^{a}O)BO₂²⁻, (R^{a}O)(R^{b}O)BO⁻, (R^{a}O)(R^{b})BO⁻, (R^{a}O)(R^{b}O)(R^{c}O)(R^{d}O)B⁻, (R^{a}O)CO₂⁻, (R^{a}O)SiO₃³⁻, (R^{a}O)(R^{b}O)SiO₂²⁻, (R^{a}O)(R^{b})SiO₂²⁻, (R^{a}O)(R^{b}O)(R^{d}O)SiO⁻, (R^{a}O)(R^{b}O)(R^{c})SiO⁻, and (R^{a}O)(R^{b})(R^{c})SiO⁻,
wherein
R^{a}, R^{b}, R^{c} and R^{d} are each, independently of one another, H, alkyl, aryl, cycloalkyl, heterocycloalkyl or heteroaryl, or
two of radicals R^{a}, R^{b}, R^{c}, and R^{d}, together with a part of the anion to which they are bound, form at least one saturated, unsaturated or aromatic ring or ring system having from 1 to 12 carbon atoms, wherein the ring or ring system can have from 1 to 5 nonadjacent heteroatoms or heteroatom-comprising groups.

9. The method according to claim 8, wherein the anion [Y]ⁿ⁻ is selected from the group consisting of compounds represented by formulae (R^{a}O)SO₃⁻, (R^{a})SO₃⁻, (R^{a}O)PO₃²⁻ and (R^{a}O)(R^{b}O)PO₂⁻, wherein R^{a} and R^{b} are each, independently of one another, alkyl, cycloalkyl, or aryl.

10. The method according to claim 9, wherein the anion [Y]ⁿ⁻ is a compound represented by formula (R^{a}O)SO₃⁻.

11. The method according to claim 1, wherein a tertiary amine or a mixture of tertiary amines is added to the ionic liquid (IL) to increase the hydrolysis stability.

12. The method according to claim 11, wherein the tertiary amine is at least one selected from the group consisting of a di(2-hydroxyethyl)(C₁-C₁₆-alkyl) amine, tri(2-hydroxyethyl)amine, an alkoxylated derivative of a di(2-hydroxyethyl)(C₁-C₁₆-alkyl)amine, and an alkoxylated derivative of tri(2-hydroxyethyl)amine.

13. The method according to claim 11, wherein the tertiary amine is a heterocyclic tertiary amine.

14. The method according to claim 13, wherein at least one ring carbon adjacent to a ring nitrogen in the heterocyclic tertiary amine comprises a substituent other than H.

15. The method according to either claim 13, wherein the heterocyclic tertiary amine is at least one selected from the group consisting of a imidazole compound, and a pyrazole compound.

16. The method according to claim 1, wherein at least one quaternary ammonium compound different from the ionic liquid (IL) is added to the ionic liquid (IL) to increase the hydrolysis stability.

17. The method according to claim 16, wherein the at least one quaternary ammonium compound comprises at least one cation selected from the group consisting of a (C₁-C₄-alkyl)(C₁-C₁₆-alkyl)-di(2 hydroxyethyl)ammonium ion, a (C₁-C₄-alkyl)tri(2-hydroxyethyl)-ammonium ion, an alkoxylated derivative of a (C₁-C₄-alkyl)(C₁-C₁₆-alkyl)-di(2 hydroxyethyl)ammonium ion, and an alkoxylated derivative of a (C₁-C₄-alkyl)tri(2-hydroxyethyl)-ammonium ion.

18. The method according to either claim 16, wherein the anion of the at least one quaternary ammonium compound is at least one C₁-C₄-alkylsulfate anion.

19. The method according to claim 1, wherein the at least one tertiary amine and/or at least one quaternary ammonium compound are added in an amount of from 0.01 to 50% by weight, based on the total weight of the ionic liquid (IL).

20. The method according to claim 2, wherein the at least one ionic liquid (IL) and the at least one tertiary amine and/or at least one quaternary ammonium compound are completely miscible with one another.
